(19) United States
(12) Patent Application Publication
(10) Pub. No.: US 2025/0201345 A1
(43) Pub. Date: Jun. 19, 2025

(54) METHOD AND ELECTRONIC DEVICE FOR PREDICTING GENE EXPRESSION FROM HISTOLOGY IMAGE BY USING ARTIFICIAL INTELLIGENCE MODEL

(71) Applicant: RESEARCH & BUSINESS FOUNDATION SUNGKYUNKWAN UNIVERSITY, Suwon-si (KR)

(72) Inventors: Young Min CHUNG, Gyeonggi-do (KR); Ji Hoon HA, Seoul (KR); Joo Sang LEE, Seoul (KR)

(73) Assignee: RESEARCH & BUSINESS FOUNDATION SUNGKYUNKWAN UNIVERSITY, Suwon-si (KR)

(21) Appl. No.: 18/970,014

(22) Filed: Dec. 5, 2024

(30) Foreign Application Priority Data
Dec. 18, 2023 (KR) 10-2023-0185082
May 28, 2024 (KR) 10-2024-0069340

(51) Int. Cl. G16B 30/20; G06T 7/00

(52) U.S. Cl. CPC G16B 30/20; G06T 7/0012; G06T 2207/20008; G06T 2207/20081; G06T 2207/30024

(57) ABSTRACT

A method, performed by an electronic device, of predicting gene expression may include obtaining global feature data corresponding to a first spot image in a histology image, obtaining local feature data corresponding to the first spot image, obtaining neighbor feature data corresponding to the first spot image, and predicting gene expression for the first spot image based on the global feature data, the local feature data, and the neighbor feature data.

### CROSS-REFERENCE TO RELATED APPLICATION

This application is based on and claims priority under 35 U.S.C. § 119 to Korean Patent Application No. 10-2023-0185082, filed on Dec. 18, 2023, in the Korean Intellectual Property Office, and Korean Patent Application No. 10-2024-0069340, filed on May 28, 2024, in the Korean Intellectual Property Office, the entire disclosures of which are incorporated herein by reference.

### BACKGROUND

### 1. Field

The disclosure relates to a method and electronic device for predicting gene expression from a histology image by using an artificial intelligence model.

This research was supported by the Samsung Future Technology Promotion Project [SRFC-MA2102-05].

### 2. Description of the Related Art

The functions of many biological systems, such as embryos, brains, or tumors, may rely on the spatial architecture of cells in tissues and spatially coordinated regulation of genes of the biological systems. In particular, cancer cells may show significantly different coordination of gene expression from their healthy counterparts. Thus, a deeper understanding of the distinct spatial organization of the cancer cells may lead to a more accurate diagnosis and treatment for cancer patients.

The recent development of the large-scale spatial transcriptome (ST) sequencing technology enables quantification of messenger ribonucleic acid (mRNA) expression of a large numbers of genes within a spatial context of tissues and cells along a predefined grid in a histology image. However, advanced ST sequencing technology may require high costs.

### SUMMARY

Additional aspects will be set forth in part in the description which follows and, in part, will be apparent from the description, or may be learned by practice of the presented embodiments of the disclosure.

The disclosure may be implemented in various ways, including a method, a system, a device, or a computer program stored in a computer-readable storage medium.

In an embodiment, there may be provided a method, performed by an electronic device, of predicting gene expression from a histology image by using an artificial intelligence model. In an embodiment, the method may include obtaining, based on a first spot image and a second spot image in the histology image, global feature data corresponding to the first spot image by using a first artificial intelligence model. In an embodiment, the method may include obtaining, from the first spot image, local feature data corresponding to the first spot image by using a second artificial intelligence model. In an embodiment, the method may include obtaining, by using a third artificial intelligence model, neighbor feature data corresponding to the first spot image based on a neighbor image including the first spot image and a surrounding region in the histology image. In an embodiment, the method may include predicting gene expression for the first spot image based on the global feature data, the local feature data, and the neighbor feature data by using a fourth artificial intelligence model.

In an embodiment, there may be provided a computer-readable recording medium having recorded thereon a program for executing, on a computer, the method of predicting gene expression from a histology image by using an artificial intelligence model.

In an embodiment, an electronic device for predicting gene expression from a histology image by using an artificial intelligence model may include a memory storing one or more instructions, and at least one processor configured to execute the one or more instructions. In an embodiment, the at least one processor may be configured to execute the one or more instructions stored in the memory to cause the electronic device to obtain, based on a first spot image and a second spot image in the histology image, global feature data corresponding to the first spot image by using a first artificial intelligence model. In an embodiment, the at least one processor may be further configured to execute the one or more instructions stored in the memory to cause the electronic device to obtain, from the first spot image, local feature data corresponding to the first spot image by using a second artificial intelligence model. In an embodiment, the at least one processor may be further configured to execute the one or more instructions stored in the memory to cause the electronic device to obtain, by using a third artificial intelligence model, neighbor feature data corresponding to the first spot image based on a neighbor image including the first spot image and a surrounding region in the histology image. In an embodiment, the at least one processor may be further configured to execute the one or more instructions stored in the memory to cause the electronic device to predict gene expression for the first spot image based on the global feature data, the local feature, data and the neighbor feature data by using a fourth artificial intelligence model.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1A is a diagram illustrating an example of predicting spot-level gene expression from a histology image, according to an embodiment;

FIG. 1B is a diagram illustrating an example of predicting spot-level gene expression from a histology image, according to an embodiment;

FIG. 1C is a diagram illustrating an example of predicting spot-level gene expression from a histology image, according to an embodiment;

FIG. 2 is a flowchart illustrating an example of a method, performed by an electronic device, of predicting spot-level gene expression from a histology image by using an artificial intelligence model, according to an embodiment;

FIG. 3 is a diagram illustrating an example of a model configured to predict spot-level gene expression from a histology image, according to an embodiment;

FIG. 4 is a diagram illustrating an image region used as a basis for an embedding model to embed a target spot image, according to an embodiment;

FIG. 5 is a diagram illustrating an example of obtaining fusion feature data based on global feature data, local feature data, and neighbor feature data, which correspond to a target spot image, according to an embodiment;

FIG. 6 is a diagram illustrating an example of obtaining global feature data corresponding to a target spot image based on initial feature data corresponding to a plurality of spot images, according to an embodiment;

FIG. 7 is a diagram illustrating an example of encoding positional information about a spot image in a histology image, according to an embodiment;

FIG. 8 is a diagram illustrating the performance of a gene expression prediction model according to an embodiment; and

FIG. 9 is a block diagram illustrating an example of an electronic device according to an embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

As the disclosure allows for various changes and numerous embodiments, particular embodiments will be illustrated in the drawings and described in detail. However, this is not intended to limit the disclosure to particular modes of practice, and it is to be appreciated that all changes, equivalents, and substitutes that do not depart from the spirit and technical scope of the disclosure are encompassed in the disclosure.

In describing embodiments, detailed descriptions of the related art will be omitted when it is deemed that they may unnecessarily obscure the gist of the disclosure. In addition, ordinal numerals (e.g., ‘first’ or ‘second’) used in the description of an embodiment are identifier codes for distinguishing one component from another. In addition, unless explicitly indicated otherwise in the context, the singular forms “a,” “an,” and “the” may be understood to include plural subjects.

Hereinafter, an embodiment of the disclosure will be described in detail with reference to the accompanying drawings to allow those of skill in the art to easily carry out the embodiment. The disclosure may, however, be embodied in many different forms and should not be construed as being limited to an embodiment set forth herein. Prior to the detailed description of the disclosure, the terms used herein may be defined or understood as follows.

In the present specification, it should be understood that when components are ‘connected’ or ‘coupled’ to each other, the components may be directly connected or coupled to each other, but may alternatively be connected or coupled to each other with a component therebetween, unless specified otherwise. In addition, ‘connection’ may refer to a wireless connection or a wired connection.

It should be understood that blocks in each flowchart, and combinations of flowcharts may be performed by one or more computer programs including computer-executable instructions. The one or more computer programs may be all stored in a single memory, or may be divided and stored in a plurality of different memories.

All functions or operations described herein may be performed by a single processor or a combination of processors. The processor or the combination of processors is a circuitry configured to perform processing, and may include circuitries such as an application processor (AP), a communication processor (CP), a graphics processing unit (GPU), a neural processing unit (NPU), a microprocessor unit (MPU), a system-on-chip (SoC), or an integrated circuit (IC).

In addition, as used herein, a component expressed as, for example, ‘ . . . er (or)’, ‘ . . . unit’, ‘ . . . module’, or the like, may denote a unit in which two or more components are combined into one component or one component is divided into two or more components according to its function. In addition, each component to be described below may additionally perform, in addition to its primary function, some or all of functions of other components take charge of, and some functions among primary functions of the respective components may be exclusively performed by other components.

As used herein, the expression ‘at least one of a, b, or c’ may refer to ‘a’, ‘b’, ‘c’, ‘both a and b’, ‘both a and c’, ‘both b and c’, ‘all of a, b, and c’, or variations thereof. As used herein, the expression ‘a or b’ may refer to ‘a’, ‘b’, ‘a and b’, or variations thereof. As used herein, the expression ‘a (b or c)’ may refer to ‘a’, ‘b’, ‘c’, ‘a and b’, ‘a and c’, ‘b and c’, ‘all of a, b and c’, or variations thereof.

In the disclosure, ‘histology image’ may include a digital image generated by photographing, with a slide scanner, a microscope, a camera, or the like, a slide that has been fixed and stained (e.g., hematoxylin and eosin (H&E)-stained) through a series of chemical processes for observing a tissue or the like removed from a human body. For example, ‘histology image’ may include a digital image captured by using a microscope, and may include information about cells, tissues, and/or structures in a human body. In an embodiment, ‘histology image’ may include a whole slide image (WSI) including a high-resolution image of a whole slide, or a pathology slide image obtained by photographing a pathology slide. In an embodiment, ‘histology image’ may refer to a part of a high-resolution WSI.

In the disclosure, ‘spot’ and ‘spot image’ may refer to a partial region of a histology image, and may be used interchangeably. For example, ‘spot’ and ‘spot image’ may include the meaning of a predefined unit region in a WSI where gene expression is quantified. For example, ‘spot’ and ‘spot image’ may include at least some of a plurality of pixels of a histology image. In an embodiment, a histology image may be segmented into a plurality of quadrangular spot images, but the shape of a spot image is not limited to a quadrangular shape. For example, the shape of a spot image may vary depending on the shape of an effective region (e.g., a tissue region or a cell region) included in the histology image. In an embodiment, a histology image may be segmented into a plurality of spot images that do not overlap each other, but the disclosure is not limited thereto. For example, a histology image may be segmented such that at least a partial region of a spot image overlaps at least a partial region of another spot image.

In the disclosure, ‘gene expression’ may refer to information about expression of one or more genes. For example, information about expression of one or more genes may include whether the one or more genes are expressed, and an expression degree, an expression level, an expression value (e.g., a messenger ribonucleic acid (mRNA) expression value), an expression pattern, and the like of the one or more genes. Thus, ‘predicting gene expression’ may refer to predicting information about an expression of one or more genes to be predicted. The one or more genes to be predicted may be genes expressed in cells or tissues of organisms, such as A2M, AKAP13, BOLA3, CDK6, C5orf38, DHX16, FAIM2, HSPB11, MED13L, MID1IP1, or ZFP36L2, but are not limited thereto. In an embodiment, genes to be predicted may vary depending on cells and/or tissues. For example, genes to be predicted for expression in a tissue of a first region of an organism, and genes to be predicted for expression in a tissue of a second region of the organism may be the same as or different from each other, or some of them are different from each other.

In the disclosure, ‘spot-level gene expression’ may include the meaning of gene expression for each spot image. In the disclosure, ‘gene expression for a spot image’ or ‘gene expression of a spot image’ may refer to gene expression at a position of a particular spot image in a histology image. For example, ‘gene expression for a spot image’, ‘spot-level gene expression’, ‘gene expression in a spot image’ or ‘gene expression information about a spot image’ may include whether one or more genes are expressed, an expression degree, an expression level, an expression value, and the like, at the position of a particular spot image in a histology image.

In the disclosure, ‘local information’, ‘local features’ or ‘local feature data’ may include microscopic information or data specific to a particular spot image in a histology image. In an embodiment, ‘local features’ or ‘local feature data’ may include information or data that may be derived from image information specific to a particular spot image. In an embodiment, ‘local information’, ‘local features’, or ‘local feature data’ may include information or data whose meaning is recognizable to a human. In an embodiment, ‘local information’, ‘local feature’, or ‘local feature data’ may include information or data whose meaning is not directly recognizable to a human. For example, ‘local feature’ or ‘local feature data’ of a particular spot image may include feature data that is output by inputting the spot image (or data regarding the spot image) into an artificial intelligence model configured to extract features from input data.

In the disclosure, ‘global context’, ‘global information’, ‘global features’, or ‘global feature data’ may include overall and macroscopic information or data regarding spot images in a histology image. In an embodiment, ‘global features’ or ‘global feature data’ is not limited to a single spot image, and may include information or data that may be derived based on information about a target spot image in a whole histology image, information about other spot images, relationship information with other spot images (e.g., correlation, similarity, or relative position), and the like. In an embodiment, ‘global context’, ‘global information’, ‘global features’, or ‘global feature data’ may include information or data whose meaning is recognizable to a human. In an embodiment, ‘global context’, ‘global information’, ‘global features’, or ‘global feature data’ may include information or data whose meaning is not directly recognizable to a human. For example, ‘global features’ or ‘global feature data’ of a particular spot image may include feature data that is output by inputting a plurality of spot images (or data regarding the plurality of spot images) into an artificial intelligence model configured to extract features from input data.

In the disclosure, ‘identifying, obtaining, extracting, deriving, calculating, predicting, determining, or generating C by using model A based on B (or from B)’ may refer to identifying, obtaining, or generating at least one of C, which is output from model A in response to receiving, as input data, at least one of B or data associated with B, or data associated with C, but is not limited thereto. For example, the data associated with B may refer to at least one of data generated or obtained by performing an arbitrary process (e.g., preprocessing), operation, or computation on B, data derived, extracted, or calculated from B, data calculated, generated, obtained, determined, or identified based on B, or part of B. For example, the data associated with C may refer to at least one of data from which C is generated or obtained by performing an arbitrary process (e.g., postprocessing), operation, or computation, data from which C is extracted, derived, or calculated, data that serves as the basis for calculating, generating, obtaining, determining, or identifying C, and data including at least part of C. In addition, in ‘identifying, obtaining, extracting, deriving, calculating, predicting, determining, or generating C by using model A based on B (or from B)’, additional other data may be input into model A in addition to at least one of B or data associated with B, and similarly, model A may also output other additional data in addition to at least one of C or data associated with C.

In the disclosure, ‘B being input into model A’ may mean not only that B is directly input into model A, but also that a result of modifying B by performing an arbitrary process on B is input into model A. In the disclosure, ‘model A outputting C’ may include not only that model A directly outputs C, but also that model A outputs data that may be modified into C through an arbitrary process.

In the disclosure, a ‘token’ may be used as a unit for processing, analyzing, or distinguishing data. For example, the term ‘token’ may be used as a unit of data that contains encapsulated particular information. In an embodiment, each token may correspond to one spot image from among a plurality of spot images. For example, global feature data corresponding to a plurality of spot images may include one or more tokens corresponding to a first spot image, and one or more tokens corresponding to a second spot image, and the one or more tokens corresponding to the first spot image may include global feature data corresponding to the first spot image. For example, one token may include vector data with d dimensions (here, d is a natural number).

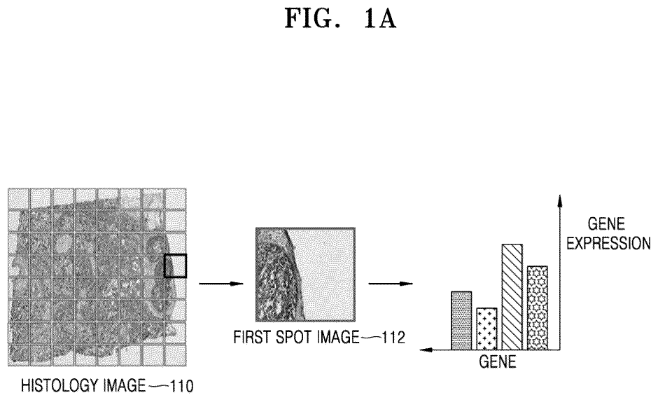

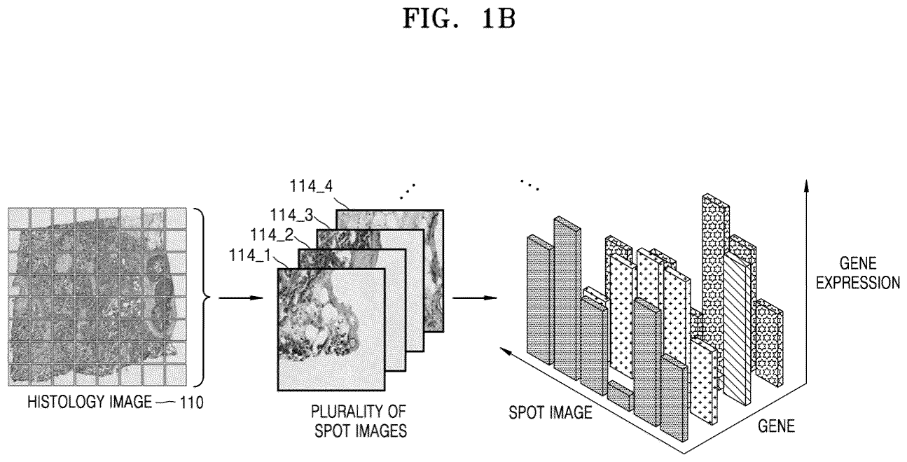

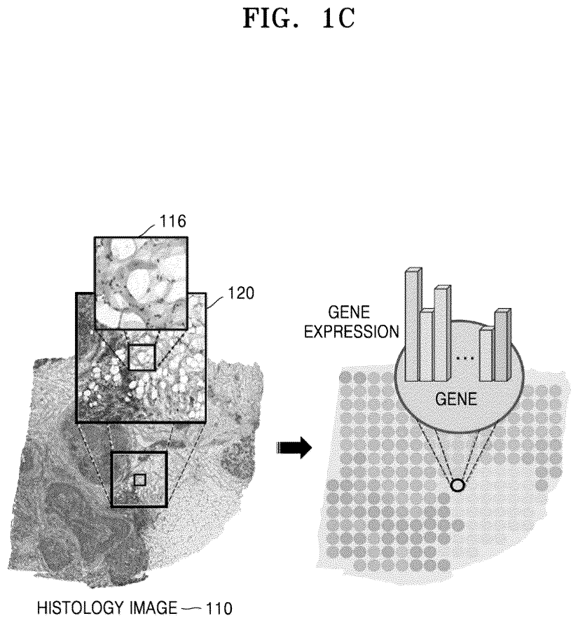

FIGS. 1A to 1C are diagrams illustrating examples of predicting spot-level gene expression from a histology image, according to an embodiment.

The emergence of large-scale spatial transcriptomics (ST) technology has facilitated the quantification of messenger ribonucleic acid (mRNA) expression across a plurality of genes within the spatial context of tissue samples. ST technology may be used to segment a centimeter-scale WSI into hundreds of thousands of spots, and provide information about gene expression for each spot. Considering the substantial costs associated with ST sequencing technology and the widespread availability of WSIs, an embodiment may provide a method of predicting spatial gene expression based on a WSI by using computer vision technology.

Various studies have been conducted to predict gene expression based on a WSI by using computer vision technology. For example, some studies may provide methods of predicting gene expression from a tissue image confined within the boundaries of a spot. For example, some other studies may provide methods of predicting gene expression by considering spatial dependencies between spot images or similarity with reference spots.

FIG. 1A is a diagram illustrating an example of predicting spot-level gene expression from a histology image 110, based on local information about spot images in the histology image 110. In an embodiment, an electronic device (e.g., at least one processor of the electronic device) may predict gene expression for a particular spot image in the histology image 110 by using only data regarding the spot image. That is, the electronic device may predict gene expression for a particular spot image in the whole histology image 110 by using only local information specific to the spot image. Referring to FIG. 1A, the electronic device may predict gene expression in a first spot image 112 from among a plurality of spot images in the histology image 110, based on only the first spot image 112. In this case, the electronic device may predict gene expression for the first spot image 112 by using only information and data included in the first spot image 112, without considering other spot images or overall information of the histology image 110.

In an embodiment, the electronic device may predict spatial gene expression patterns in individual spot images in the histology image 110 (or a WSI) by using a pre-trained convolutional neural network (CNN)-based model. For example, the electronic device may predict a gene expression value for each spot image by segmenting the histology image 110 into a plurality of spot images and inputting each of the spot images as one input into an artificial intelligence model. For example, the artificial intelligence model operating in the electronic device may receive each spot image as individual input data and output a gene expression prediction result for the spot image. That is, the first spot image 112 may not affect gene expression prediction results for other spot images, and similarly, a gene expression prediction result for the first spot image 112 may not be affected by the other spot images.

FIG. 1B is a diagram illustrating an example of predicting spot-level gene expression from the histology image 110, based on global information about spot images in the histology image 110. In an embodiment, the electronic device may predict gene expression for a plurality of spot images 114_1, 114_2, 114_3, 114_4, . . . (e.g., all spot images) in the histology image 110, by using data regarding the plurality of spot images 114_1, 114_2, 114_3, 114_4, . . . together. That is, the electronic device may predict gene expression for the spot images in the histology image 110 based on a global context (or global information) of the spot images. Referring to FIG. 1B, the electronic device may predict gene expression in the spot images in the histology image 110 by simultaneously considering the plurality of spot images 114_1, 114_2, 114_3, 114_4, . . . .

In an embodiment, the electronic device may simultaneously predict spatial gene expression patterns from the plurality of spot image 114_1, 114_2, 114_3, 114_4, . . . (e.g., all spot images) in the histology image 110 (or a WSI) by using a vision transformer (ViT)-based model. For example, the electronic device may predict a gene expression value for each spot image by segmenting the histology image 110 into the plurality of spot images 114_1, 114_2, 114_3, 114_4, . . . , and inputting data regarding the plurality of spot images 114_1, 1142, 114_3, 114_4, . . . as one input into an artificial intelligence model. For example, the artificial intelligence model operating in the electronic device may receive the plurality of spot images 114_1, 114_2, 114_3, 114_4, . . . as input data, and output gene expression prediction results for the spot images. That is, an arbitrary spot image may affect gene expression prediction results for other spot images.

When prediction is performed by directly inputting the plurality of spot images 114_1, 114_2, 114_3, 114_4, . . . with large data sizes into a large-scale artificial intelligence model, the computational burden on the electronic device may increase. Accordingly, in an embodiment, the electronic device may extract initial feature data from each of the plurality of spot images 114_1, 114_2, 114_3, 114_4, . . . , and use the extracted initial feature data as input data for the artificial intelligence model. For example, the electronic device may extract, from the first spot image, initial feature data regarding the first spot image, and extract, from a second spot image, initial feature data regarding the second spot image. The electronic device may predict gene expression in each of the spot images by inputting the extracted initial feature data regarding the first spot image, the extracted initial feature data regarding the second spot image, . . . , and the extracted initial feature data regarding an n-th spot image, as one input data into the artificial intelligence model.

Meanwhile, because local properties such as the morphology of cells or cell organelles may be directly associated with gene expression, fine-grained local information may be important in predicting gene expression. In addition, because gene expression in a cell is highly correlated with gene expression in other cells in a tissue, a global context may also be important in predicting gene expression. Thus, when prediction is performed based on only local information about a spot image without considering information about other spot images and overall information about a histology image, abundant biological information in a wider range of image contexts cannot be considered, and thus, the performance of gene expression prediction may be poor. On the contrary, even when prediction is performed by focusing on only a global context, detailed information included in local information of a spot image cannot be considered, and thus, the performance of gene expression prediction may be poor. Thus, in one or more of the embodiments described above, the performance of gene expression prediction may be poor because prediction is performed by focusing on only local information or a global context.

In order to improve the performance of gene expression prediction, an embodiment may provide a deep learning framework designed to leverage multi-resolution features of a histology image. For example, the electronic device may extract three types of features corresponding to a target spot image based on images with different resolutions (or sizes) in a histology image, and predict gene expression for a target spot image by using the extracted features. For example, the three types of features corresponding to a target spot image may include features based on the target spot image (e.g., a spot to be predicted for gene expression), features based on a neighbor image including the target spot image (e.g., an image further including the target spot and its surrounding regions), and features based on a plurality of spot images, and accordingly, may encompass biological information at various levels, ranging from the detailed cell morphology in the target spot image to the surrounding tissue phenotype and the overall tissue microenvironment in the WSI.

FIG. 1C is a diagram illustrating an example of predicting spot-level gene expression from the histology image 110, based on local information, neighbor information, and global information about spot images in the histology image 110. In an embodiment, in order to improve the performance of gene expression prediction, the electronic device may integrate local information, neighbor information, and global information about spot images, and predict spot-level gene expression based on the integrated information. Referring to FIG. 1C, the electronic device may extract, from a second spot image 116 in the histology image 110, local features of the second spot image 116, extract, from a neighbor image 120 including the second spot image 116, neighbor features of the second spot image 116, and extract global features of the second spot image 116 based on the plurality of spot images 114_1, 114_2, 1143, 114_4, . . . . The electronic device may predict gene expression for the second spot image 116 by using all of the local features of the second spot image 116, the neighbor features of the second spot image 116, and the global features of the second spot image 116. For example, the electronic device may integrate the local features, the neighbor features, and the global features of the second spot image 116, and predict gene expression for the second spot image 116 based on the integrated features.

According to an embodiment, the electronic device may improve the prediction accuracy while preventing excessive computational costs, by utilizing information about images having various resolutions for a target spot image. According to an embodiment, the electronic device may improve the performance of gene expression prediction while minimizing additional computing costs, by integrating various biological contexts to predict a spatial gene expression level from a WSI.

In FIGS. 1A and 1C, the prediction results show gene expression values for respective genes in the form of a two-dimensional bar graph, but the method of expressing a prediction result and the form of the prediction result are not limited thereto. In addition, in FIG. 1B, the prediction result shows gene expression values for respective genes in each spot image in the form of a three-dimensional bar graph, but the method of expressing a prediction result and the form of the prediction result are not limited thereto.

In addition, in FIGS. 1A and 1B, the prediction results include expression values of four types of genes, but the number of genes to be predicted is not limited thereto.

The plurality of spot image 114_1, 1142, 114_3, 114_4, . . . of FIGS. 1B and 1C may include at least one of the first spot image 112 of FIG. 1A or the second spot image 116 of FIG. 1C.

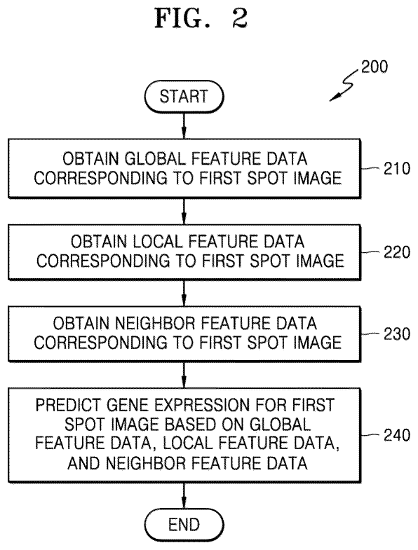

FIG. 2 is a flowchart illustrating an example of a method, performed by an electronic device, of predicting spot-level gene expression from a histology image by using an artificial intelligence model, according to an embodiment.

In describing FIG. 2, redundant descriptions provided above with reference to any one of FIGS. 1A to 1C may be omitted.

Referring to FIG. 2, a method 200 of predicting spot-level gene expression from a histology image by using an artificial intelligence model according to an embodiment may include operations 210 to 240. In an embodiment, operations 210 to 240 may be executed by at least one processor included in the electronic device. In one or more embodiments, the method 200, performed by an electronic device, of predicting spot-level gene expression from a histology image by using an artificial intelligence model is not limited to that illustrated in FIG. 2, and may further include operations not illustrated in FIG. 2, or may not include some of the operations illustrated in FIG. 2.

In operation 210, the electronic device (e.g., a processor of the electronic device) may obtain, based on a first spot image and a second spot image in a histology image, global feature data corresponding to the first spot image by using a first artificial intelligence model. For example, the first artificial intelligence model may include a global feature extraction model or a global embedding model that is trained to extract or output global feature data regarding a spot image.

In an embodiment, the electronic device may obtain first initial feature data from the first spot image, and second initial feature data from the second spot image, by using a pre-trained model. The electronic device may obtain global feature data corresponding to the first spot image based on the obtained first initial feature data and second initial feature data, by using the first artificial intelligence model. In an embodiment, the electronic device may obtain global feature data corresponding to the first spot image, wherein positional information about the first spot image is encoded in the global feature data, by using the first artificial intelligence model. For example, the first artificial intelligence model may include a positional information encoding model configured to encode positional information in a histology image.

In operation 220, the electronic device may obtain, from the first spot image, local feature data corresponding to the first spot image by using a second artificial intelligence model. For example, the electronic device may obtain local feature data corresponding to the first spot image based on information limited to a range of a target spot image, without considering other image regions in the histology image. For example, the second artificial intelligence model may include a local feature extraction model or a local embedding model that is trained to extract or output local feature data regarding a spot image.

In operation 230, the electronic device may obtain neighbor feature data corresponding to the first spot image based on a neighbor image including the first spot image in the histology image, by using a third artificial intelligence model. In an embodiment, the electronic device may obtain, from the neighbor image, initial feature data corresponding to the neighbor image by using a pre-trained model, and may obtain neighbor feature data corresponding to the first spot image from the initial feature data corresponding to the neighbor image, by using a third artificial intelligence model. For example, the electronic device may segment the neighbor image into a plurality of sub-regions including a first sub-region and a second sub-region, and use a pre-trained model to obtain, from the first sub-region, third initial feature data corresponding to the first sub-region, and to obtain, from the second sub-region, fourth initial feature data corresponding to the second sub-region. The initial feature data corresponding to the neighbor image may include the third initial feature data corresponding to the first sub-region, and the fourth initial feature data corresponding to the second sub-region.

In operation 240, the electronic device may predict gene expression for the first spot image based on the global feature data, the local feature data, and the neighbor feature data, which correspond to the first spot image, by using a fourth artificial intelligence model. In an embodiment, the electronic device may obtain fusion feature data corresponding to the first spot image, based on the global feature data, the local feature data, and the neighbor feature data by using a fifth artificial intelligence model. The electronic device may predict gene expression for the first spot image based on the fusion feature data corresponding to the first spot image by using the fourth artificial intelligence model.

In an embodiment, the electronic device may use the fifth artificial intelligence model to obtain global-neighbor fusion feature data corresponding to the first spot image based on the global feature data and the neighbor feature data, and to obtain global-local fusion feature data corresponding to the first spot image based on the global feature data and the local feature data. The electronic device may predict gene expression for the first spot image based on the global-neighbor fusion feature data and the global-local fusion feature data. For example, the electronic device may obtain fusion feature data corresponding to the first spot image based on the global-neighbor fusion feature data and the global-local fusion feature data, and predict gene expression for the first spot image based on the fusion feature data by using the fourth artificial intelligence model.

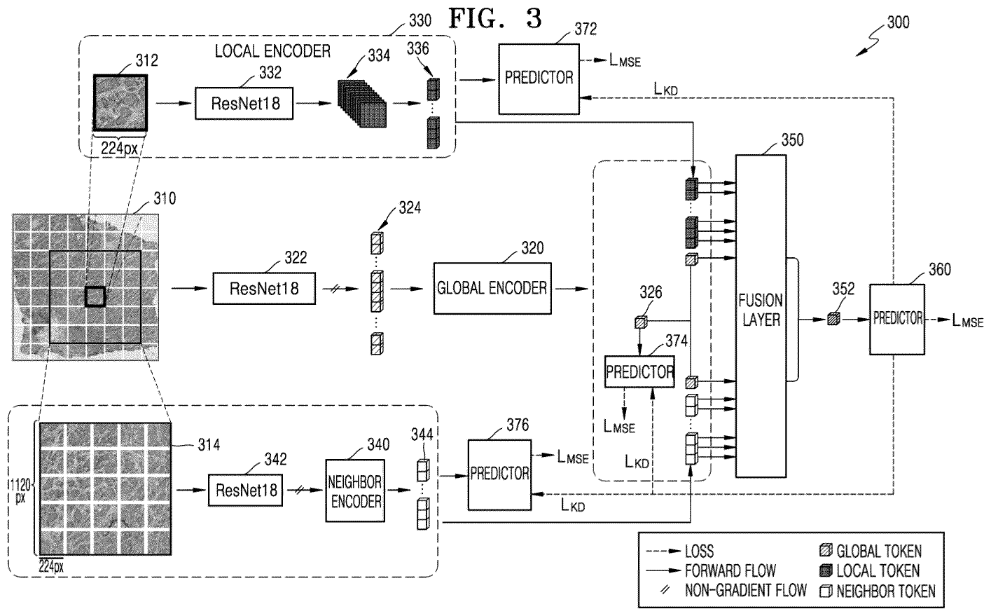

FIG. 3 is a diagram illustrating an example of a model configured to predict spot-level gene expression from a histology image, according to an embodiment.

In describing FIG. 3, redundant descriptions provided above with reference to any one of FIGS. 1A to 2 may be omitted.

Due to arbitrary shapes in tissue biopsy, a histology image 310 is not easily regularized into a square shape, unlike general images that may be used in existing image network models (e.g., ImageNet) or other general resources. The histology image 310 has different characteristics from general images, and thus, models and methods used for analysis of general images may not be appropriate for analyzing the histology image 310. Thus, it may be difficult to achieve robust prediction, that is, high prediction performance, in predicting gene expression from the histology image 310 by using existing models and existing methods.

FIG. 3 may represent an artificial intelligence model (hereinafter, a ‘gene expression prediction model’) 300 designed to predict spot-level gene expression from a histology image 310, according to an embodiment. In an embodiment, the electronic device may embed each of local features, neighbor features, and global features, which correspond to a target spot image 312, and utilize all of the three types of features to predict gene expression for the target spot image 312 in the histology image 310. For example, the electronic device may obtain each feature of the target spot image 312 by using a global embedding model, a local embedding model, and a neighbor embedding model that are included in the gene expression prediction model 300. For example, the electronic device may fuse three types of features corresponding to the target spot image 312, and predict gene expression for the target spot image 312 from the fused features.

Referring to FIG. 3, different independent encoders may be used to extract the respective types of features corresponding to the target spot image 312, and the types of features extracted by the respective encoders may be integrated through a fusion layer, for effective prediction of gene expression. For example, the gene expression prediction model 300 may include a global encoder 320, a local encoder 330, a neighbor encoder 340, and a fusion layer 350, and more detailed information about the target spot image 312 may be captured by the local encoder 330 and the neighbor encoder 340. For example, an encoder may include hardware modules, software modules, or a combination thereof. For example, different encoders may include different hardware modules, or may include different software modules.

The global encoder 320 of the gene expression prediction model 300 may include a global embedding model (or a global feature extraction model) configured to extract global feature data by embedding global information about a spot image. The local encoder 330 of the gene expression prediction model 300 may include a local embedding model (or a local feature extraction model) configured to extract local feature data by embedding local information about a spot image. The neighbor encoder 340 of the gene expression prediction model 300 may include a neighbor embedding model (or a neighbor feature extraction model) configured to extract neighbor feature data by embedding information about a spot image and a surrounding region. Thus, the gene expression prediction model 300 may include, as sub-models, the global embedding model (or global feature extraction model), the local embedding model (or local feature extraction model), and the neighbor embedding model (or neighbor feature extraction model).

Hereinafter, a method of predicting gene expression for the target spot image 312 based on the gene expression prediction model 300 according to an embodiment will be described.

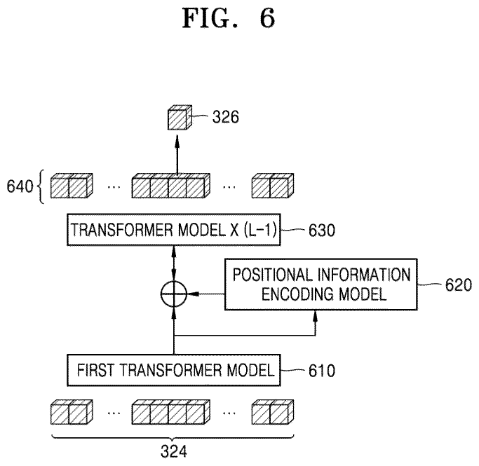

In an embodiment, the electronic device may obtain (identify, generate, calculate, determine, extract, or derive), based on a plurality of spot images in the histology image 310, global feature data corresponding to each of the plurality of spot images by using the global embedding model. For example, the global embedding model may receive, as input, data associated with the plurality of spot images, and output (calculate, extract, derive, determine, or generate) global features of each spot image within the plurality of spot images. For example, the global embedding model may capture global context-aware features from spot images (e.g., all spot images) in the histology image 310 (e.g., a WSI). The global embedding model may include one or more transformer models (blocks or layers), and an embodiment of the global embedding model may be described below with reference to FIG. 6.

The histology image 310 may include a plurality of spot images with a predetermined (predefined or preset) size (or resolution). For example, the histology image 310 may be a high-resolution image with a large data size, and may include hundreds to thousands of spot images with a size of 224×224. Inputting the histology image 310 itself or the plurality of spot images with a size of 224×224 as a single piece of input data into the global embedding model may place an excessive computational burden on the electronic device.

To reduce the computational burden on the electronic device where the global embedding model operates, a process for data dimensionality reduction (e.g., low-dimensional embedding) may be performed on each spot image. In an embodiment, the electronic device may obtain (identify, generate, calculate, determine, extract, or derive) initial feature data corresponding to each spot image, from the plurality of spot images in the histology image 310 by using a pre-trained model. For example, the pre-trained model may receive each of a plurality of spot images, as independent individual input data, and output (output, extract, derive, determine, or generate) initial feature data corresponding to the spot image. For example, the pre-trained model may receive, as input, a first spot image and output initial feature data corresponding to the first spot image, and receive, as input, a second spot image and output initial feature data corresponding to the second spot image. Thus, when the pre-trained model outputs the initial feature data corresponding to the first spot image, information about the second spot image may not be considered.

In an embodiment, the electronic device may obtain (identify, generate, calculate, determine, extract, or derive) global feature data corresponding to each spot image, based on initial feature data corresponding to the plurality of spot images by using the global embedding model. For example, the global embedding model may receive the initial feature data corresponding to the plurality of spot images as a single piece of input data, and output (calculate, extract, derive, determine, or generate) global feature data corresponding to each of the plurality of spot images. For example, the global embedding model may receive, as input, initial feature data corresponding to a first spot image and initial feature data corresponding to a second spot image such that the pieces of data are associated with each other, and output global feature data corresponding to the first spot image, and global feature data corresponding to the second spot image. Thus, in outputting the global feature data corresponding to the first spot image, the global embedding model may consider not only the initial feature data corresponding to the first spot image but also initial feature data corresponding to other spot images (e.g., the second spot image).

The global encoder 320 illustrated in FIG. 3 may include the global embedding model, may be composed of the global embedding model, or may be the global embedding model itself. For example, referring to FIG. 3, a pre-trained ResNet-18 model 322 may receive a plurality of spot images in the histology image 310 as input, and output initial tokens 324 corresponding to the plurality of spot images. The spot images and the initial tokens 324 may correspond to each other, respectively. Thus, the pre-trained ResNet-18 model 322 may output as many initial tokens 324 as the number of input spot images.

The global encoder 320 may receive the initial tokens 324 corresponding to the plurality of spot images, and output global tokens corresponding to the plurality of spot images. The global encoder 320 may output as many global tokens as the number of input initial tokens 324 (or the number of spot images), but in order to illustrate an example of the process of predicting gene expression for the target spot image 312, FIG. 3 may illustrate only a global token 326 corresponding to the target spot image 312. For example, a global token corresponding to each spot image may be composed of d-dimensional data (d is a predetermined natural number), and the global token 326 corresponding to n spot images (n is a natural number) may include n d-dimensional tokens.

In an embodiment, the electronic device may obtain (identify, generate, calculate, determine, extract, or derive), from the target spot image 312, local feature data corresponding to the target spot image 312 by using the local embedding model. For example, the local embedding model may output (calculate, extract, derive, determine, or generate) local feature data corresponding to the target spot image 312 based on only information limited to the target spot image 312 without considering information about other spot images in the histology image 310. For example, the local embedding model may include a model that is fine-tuned to capture fine-grained local features from each spot image. For example, the local embedding model may include a ResNet-18 model 332, and components following global average pooling may be omitted. The ResNet-18 model 332 included in the local embedding model may be a pre-trained model, but may be dynamically updated during a process of training the gene expression prediction model 300. For example, the local embedding model may embed the target spot image 312 having a size of 224×224 into 49 distinct features, and each feature may be composed of 512-dimensional data.

The local encoder 330 illustrated in FIG. 3 may include the local embedding model, may be composed of the local embedding model, or may be the local embedding model itself. For example, referring to FIG. 3, the local encoder 330 including the ResNet-18 model 332 may output, from the target spot image 312, local token(s) 336 corresponding to the target spot image 312. For example, the local encoder 330 may output nₗₒ d-dimensional tokens (nₗₒ is a natural number) for one spot image. For example, referring to FIG. 3, the ResNet-18 model 332 may receive a target spot image 312 having a size of 224×224 as input, and output 512 7×7 feature maps, and the local encoder 330 may reshape the output 512 7×7 feature maps into 49 512-dimensional local tokens 336.

In an embodiment, the electronic device may obtain (identify, generate, calculate, determine, extract, or derive) neighbor feature data corresponding to the target spot image 312, based on a neighbor image 314 including the target spot image 312 by using the neighbor embedding model. The neighbor embedding model may extract features that reflect biological information or context in a relatively wide range, including not only the target spot image 312 but also its surrounding region. For example, the neighbor embedding model may capture a correlation between the target spot image 312 and its surrounding region. For example, the neighbor embedding model may include one or more self-attention models (blocks or layers) or relative position encoding models (blocks or layers) configured to perform self-attention computation based on input data.

The neighbor image 314 may be an image having a predetermined size and including the target spot image 312 and its surrounding region. For example, the neighbor image 314 may not be a set of other spot images adjacent to the target spot image 312, but may be an image including regions directly adjacent to the target spot image 312. For example, the neighbor image 314 may be an image having a size of 1120×1120 and including the target spot image 312 and its surrounding region. Inputting the neighbor image 314 that has a relatively large size as input data directly into the neighbor embedding model requires a lot of resources and may increase the computational burden on the electronic device.

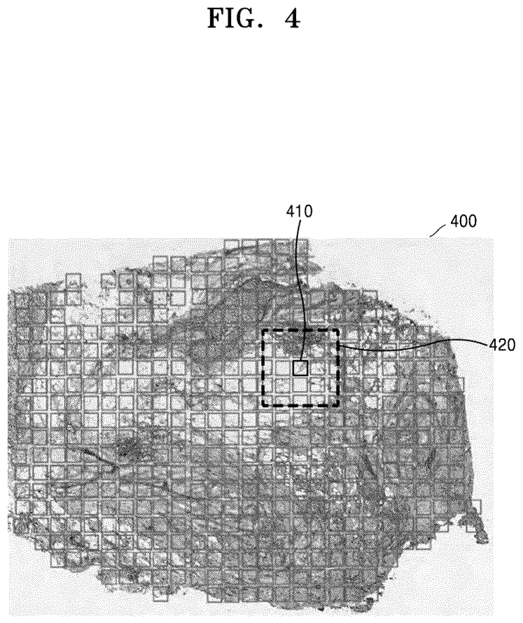

To reduce the computational burden on the electronic device where the neighbor embedding model operates, a process for data dimensionality reduction (e.g., low-dimensional embedding) may be performed on the neighbor image 314. In an embodiment, the electronic device may obtain, from the neighbor image 314, initial feature data corresponding to the neighbor image 314 by using a pre-trained model. For example, the neighbor image 314 may include a plurality of sub-regions, and the initial feature data corresponding to the neighbor image 314 may include initial feature data corresponding to each sub-region. An embodiment of the neighbor image 314 may be described below with reference to FIG. 4.

In an embodiment, the electronic device may segment the neighbor image 314 into sub-regions having a predetermined size, and obtain, from a plurality of sub-regions included in the neighbor image 314, initial feature data corresponding to each sub-region by using a pre-trained model. For example, the pre-trained model may receive each of the plurality of sub-regions as independent individual input data, and output initial feature data corresponding to the sub-region. For example, the pre-trained model may input a first sub-region as input and output initial feature data corresponding to the first sub-region, and input a second sub-region as input and output initial feature data corresponding to the second sub-region.

In an embodiment, the electronic device may obtain (identify, generate, calculate, determine, extract, or derive) neighbor feature data corresponding to the target spot image 312 based on initial feature data corresponding to the neighbor image 314, by using the neighbor embedding model. For example, the neighbor embedding model may receive the initial feature data corresponding to the neighbor image 314 as input data, and output (calculate, extract, derive, determine, or generate) neighbor feature data corresponding to the target spot image 312. For example, the neighbor embedding model may receive initial feature data corresponding to the sub-regions of the neighbor image 314 as one piece of input data, and output neighbor feature data corresponding to the target spot image 312.

The neighbor encoder 340 illustrated in FIG. 3 may include the neighbor embedding model, may be composed of the neighbor embedding model, or may be the neighbor embedding model itself. For example, referring to FIG. 3, a pre-trained ResNet-18 model 342 may receive the neighbor image 314 having a size of 1120×1120 as input, and output a feature vector corresponding to the neighbor image 314. For example, the neighbor image 314 having a size of 1120×1120 may be segmented into 25 sub-regions having a size of 224×224, and the pre-trained ResNet-18 model 342 may input each sub-region as independent individual input data, and output a 512-dimensional feature vector corresponding to the sub-region. The sub-regions and the output feature vectors may correspond to each other, respectively. Thus, the pre-trained ResNet-18 model 342 may output 25 512-dimensional feature vectors from 25 sub-regions.

The neighbor encoder 340 may receive, as input, the feature vectors corresponding to the sub-regions, and output neighbor token(s) 334 corresponding to the target spot image 312. For example, the neighbor encoder 340 may output nₙₑ d-dimensional neighbor tokens (nₙₑ is a natural number) for one spot image. For example, the neighbor encoder 340 may output, for one spot image, as many d-dimensional neighbor tokens as the number of sub-regions included in the neighbor image 314. Referring to FIG. 3, the neighbor encoder 340 may receive 25 512-dimensional feature vectors as input, and output 25 512-dimensional neighbor tokens 344 corresponding to the target spot image 312.

Features output independently from the respective encoders may be appropriately integrated for gene expression prediction. In an embodiment, the electronic device may obtain (identify, generate, calculate, determine, extract, or derive) fusion feature data corresponding to the target spot image 312 based on global feature data, local feature data, and neighbor feature data, which correspond to the target spot image 312, by using a feature fusion model. For example, the electronic device may use the feature fusion model to obtain global-local fusion feature data corresponding to the target spot image 312 based on the global feature data and the local feature data, and to obtain global-neighbor fusion feature data corresponding to the target spot image 312 based on the global feature data and the neighbor feature data. For example, the electronic device may obtain fusion feature data corresponding to a first spot image based on the global-local fusion feature data and the global-neighbor fusion feature data. For example, the electronic device may obtain fusion feature data corresponding to the first spot image by performing an element-wise sum or an element-wise weighted sum on the global-local fusion feature data and the global-neighbor fusion feature data.

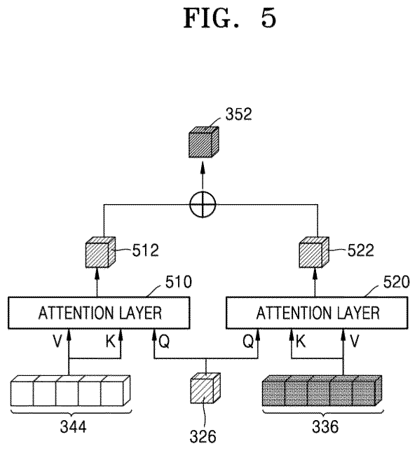

The gene expression prediction model 300 according to an embodiment may include a feature fusion model as a sub-model, and the feature fusion model as a sub-model may be included in the gene expression prediction model 300 in the form of one or more layers (or blocks) as illustrated in FIG. 3. For example, the feature fusion model may receive, as input, global feature data, local feature data, and neighbor feature data corresponding to the target spot image 312, and output (calculate, extract, derive, determine, or generate) fusion feature data corresponding to the target spot image 312. For example, the feature fusion model may receive, as input, global feature data, local feature data, and neighbor feature data corresponding to the target spot image 312, and output global-local fusion feature data and global-neighbor fusion feature data corresponding to the target spot image 312. For example, the feature fusion model may include one or more attention layers (or blocks), and an embodiment of the feature fusion model may be described below with reference to FIG. 5.

The fusion layer 350 illustrated in FIG. 3 may include the feature fusion model, may be composed of the feature fusion model, or may be the feature fusion model itself. For example, referring to FIG. 3, the fusion layer 350 may receive, as input, the global token 326, the local tokens 336, and the neighbor tokens 344 corresponding to the target spot image 312, and output a fusion token 352 corresponding to the target spot image 312. For example, the fusion layer 350 may output a global-local fusion token based on the global token 326 and the local tokens 336, and may output a global-neighbor fusion token based on the global token 326 and the neighbor tokens 344, and the global-neighbor fusion token and the global-local fusion token may be integrated into the fusion token 352 with d (e.g., 512) dimensions. For example, the fusion layer 350 may independently perform global-local fusion and global-neighbor fusion, and may integrate a global token with neighbor tokens, and integrate the global token with local tokens.

In an embodiment, the electronic device may predict gene expression for the target spot image 312, based on the fusion feature data corresponding to the target spot image 312 by using a final prediction model. For example, the electronic device may obtain (identify, generate, calculate, determine, extract, or derive) a gene expression prediction value (or level) for one or more genes in the target spot image 312, based on the fusion feature data corresponding to the target spot image 312 by using the final prediction model. The gene expression prediction model 300 according to an embodiment may include the final prediction model as a sub-model, and the final prediction model as a sub-model may be included in the gene expression prediction model 300 in the form of a predictor (layer or block) as illustrated in FIG. 3. For example, the final prediction model may receive the fusion feature data corresponding to the target spot image 312 as input, and output (calculate, extract, derive, determine, or generate) a gene expression prediction value for the target spot image 312. For example, the final prediction model may include a fully-connected layer.

A predictor 360 illustrated in FIG. 3 may include the final prediction model, may be composed of the final prediction model, or may be the final prediction model itself. For example, referring to FIG. 3, the predictor 360 may receive, as input, the fusion token 352 corresponding to the target spot image 312, and predict gene expression for the target spot image 312. For example, the predictor 360 may receive, as input, the d-dimensional fusion token 352 corresponding to the target spot image 312, and output an expression value (or level) predicted for each of one or more genes (e.g., m genes) in the target spot image 312.

Hereinafter, a method of training or updating the gene expression prediction model 300 by using training data according to an embodiment will be described.

In an embodiment, at least some of the global embedding model, the local embedding model, the neighbor embedding model, the feature fusion model, or the final prediction model may be included as sub-models in the gene expression prediction model 300, and may be connected to each other in an end-to-end manner to be trained or updated. As illustrated in FIG. 3, flows of the global encoder 320, the local encoder 330, the neighbor encoder 340, the fusion layer 350, and the predictor 360 correspond to a forward flow of the gene expression prediction model 300 and may correspond to a gradient flow. Thus, the global encoder 320, the local encoder 330, the neighbor encoder 340, the fusion layer 350, and the predictor 360 may be dynamically trained or updated in a process of training the gene expression prediction model 300. For example, the global encoder 320, the local encoder 330, the neighbor encoder 340, the fusion layer 350, and the predictor 360 may be trained or updated according to a predetermined loss function.

In an embodiment, in order to reduce the computational burden on the electronic device, pre-trained models used for data dimensionality reduction (e.g., the pre-trained ResNet-18 model 322 and the pre-trained ResNet-18 model 342 of FIG. 3) may not be dynamically updated even when training the gene expression prediction model 300, and may be fixed. For example, the pre-trained models may include a model that has been completely trained through self-supervised learning by using histology images from various sources. As illustrated in FIG. 3, the flow through which the ResNet-18 model 322 extracts the initial tokens 324 from a plurality of spot images corresponds to a non-gradient flow, and the ResNet-18 model 322 may have fixed weight values that are not dynamically updated during a process of training or updating the gene expression prediction model 300. In addition, as illustrated in FIG. 3, the flow through which the ResNet-18 model 342 extracts feature vectors from sub-regions of the neighbor image 314 corresponds to a non-gradient flow, and the ResNet-18 model 342 may have fixed weight values that are not dynamically updated during a process of training or updating the gene expression prediction model 300.

In an embodiment, the pre-trained model used for data dimensionality reduction of a spot image, and the pre-trained model used for data dimensionality reduction of a sub-region may be the same as or different from each other. For example, the pre-trained model for a spot image and the pre-trained model for a sub-region may have the same or different structures. For example, the pre-trained model for a spot image and the pre-trained model for a sub-region may be configured with different weight values.

In an embodiment, the electronic device may train or update the gene expression prediction model 300 by using training data. The electronic device that trains the gene expression prediction model 300 and an electronic device that uses the gene expression prediction model 300 (or an electronic device on which the gene expression prediction model 300 operates) may be the same as or different from each other.

In an embodiment, the gene expression prediction model 300 may be trained based on a predetermined (predefined or preset) loss function. For example, the gene expression prediction model 300 may be trained or updated such that the value of a loss based on a loss function is minimized. For example, the electronic device may update at least one weight of the gene expression prediction model 300 such that the value of a total loss calculated according to a loss function is minimized. According to an embodiment, a loss function based on a fusion loss mechanism may be determined (defined or set) to optimize information integration of various types of features (or tokens) in a process of training the gene expression prediction model 300.

In an embodiment, the gene expression prediction model 300 may be trained or updated by using a loss function based on a difference between a gene expression value predicted for a target spot image included in a training image (hereinafter, referred to as a “training target spot image”), and a ground-truth gene expression value for the target spot image. For example, the electronic device may train or update the gene expression prediction model 300 based on Equation (1) below.

$${L}^{F} = \frac{1}{m} {\sum }_{k = 1}^{m} {‖ {q}_{k}^{F} - {y}_{k} ‖}_{2}^{2} \qquad ( 1 )$$

In Equation (1), L^{F} may denote a loss based on a difference between a value predicted from fusion feature data, and a ground-truth (hereinafter, referred to as a first loss L_{MSE}), m may denote the number of genes to be predicted, qₖ^{F} may denote a value for expression of a k-th gene predicted from fusion feature data, and yₖ may denote a ground-truth value for expression of the k-th gene. Thus, the gene expression prediction model 300 may be trained by using a mean squared error (MSE) loss based on a gene expression value predicted from fusion feature data, and a ground-truth gene expression value.

In an embodiment, the electronic device may train or update an artificial intelligence model by using a loss function that is determined (defined, set, or designed) to improve the individual performance of each encoder. For example, the gene expression prediction model 300 may be trained or updated by using a fusion loss mechanism based on knowledge distillation. For example, the electronic device may utilize gene expression-related information inherent in fusion tokens to improve the performance of the global encoder 320, the local encoder 330, and the neighbor encoder 340.

In an embodiment, the gene expression prediction model 300 may be trained by using a loss function based on a difference between at least one of a gene expression value predicted from local feature data corresponding to a training target spot image, a gene expression value predicted from global feature data corresponding to the training target spot image, or a gene expression value predicted from neighbor feature data corresponding to the training target spot image, and a gene expression value predicted from fusion feature data corresponding to the training target spot image. In an embodiment, the gene expression prediction model 300 may be trained by using a loss function based on a difference between at least one of a gene expression value predicted from local feature data corresponding to a training target spot image, a gene expression value predicted from global feature data corresponding to the training target spot image, or a gene expression value predicted from neighbor feature data corresponding to the training target spot image, and a ground-truth gene expression value for the training target spot image. For example, the loss function for training the gene expression prediction model 300 may include a loss due to a difference between each of a gene expression prediction value from local feature data, a gene expression prediction value from global feature data, and a gene expression prediction value from neighbor feature data, and a gene expression prediction value from fusion feature data (hereinafter, referred to as a second loss L_{KD}), and a loss due to a difference between each of a gene expression prediction value from local feature data, a gene expression prediction value from global feature data, and a gene expression prediction value from neighbor feature data and a ground-truth gene expression value (hereinafter, referred to as a third loss L_{KD}).

For example, the electronic device may train or update the gene expression prediction model 300 based on Equation (2) below.

$${L}^{j} = ( 1 - α ) \frac{1}{m} {\sum }_{k = 1}^{m} {‖ {q}_{k}^{j} - {y}_{k} ‖}_{2}^{2} + α \frac{1}{m} {\sum }_{k = 1}^{m} {‖ {q}_{k}^{j} - {q}_{k}^{F} ‖}_{2}^{2} \qquad ( 2 )$$

In Equation (2), L^{j} may denote a loss for the j-th token, the first token may refer to a global token, the second token may refer to a local token, and the third token may refer to a neighbor token. In addition, qₖ^{j} may denote a predicted value for expression of the k-th gene based on the j-th token, yₖ may denote a ground-truth value for expression of the k-th gene, and qₖ^{F} may denote a predicted value for expression of the k-th gene based on a fusion token. α may denote a parameter for adjusting the importance between the second loss and the third loss.

In order to train or update the gene expression prediction model 300 based on the loss function described above, as illustrated in FIG. 3, a predictor configured to predict gene expression based on output data from each encoder may be connected to or associated with each encoder. For example, a first predictor 372 may be used to predict a gene expression value from local feature data corresponding to a training target spot image, a second predictor 374 may be used to predict a gene expression value from global feature data corresponding to the training target spot image, and a third predictor 376 may be used to predict a gene expression value from neighbor feature data corresponding to the training target spot image. For example, each predictor may include a fully-connected layer, and may predict gene expression based on input data. In a process of training or updating the gene expression prediction model 300, each predictor may be dynamically trained or updated, or may have fixed weights without being trained or updated.

For example, the first predictor 372 connected to (or associated with) the local encoder 330 or the third predictor 376 connected to (or associated with) the neighbor encoder 340 may include average pooling layers (or blocks), and the average pooling layers (or blocks) may perform an average pooling operation on input tokens. Thus, the electronic device may perform an average pooling operation on the local tokens 336 by using the first predictor 372, and obtain a gene expression prediction value based on the local tokens 336 from a result of the average pooling operation. Similarly, the electronic device may perform an average pooling operation on the neighbor tokens 344 by using the third predictor 376, and obtain a gene expression prediction value based on the neighbor tokens 344 from a result of the average pooling operation.

In an embodiment, the electronic device may train or update the gene expression prediction model 300 based on the loss function including all of the first loss, the second loss, and the third loss described above. For example, the electronic device may train an artificial intelligence model by using Equation (3) below.

$$L = {\sum }_{j}^{3} {L}^{j} + {L}^{F} \qquad ( 3 )$$

In Equation (3), L^{j} may denote a loss for the j-th token calculated according to Equation (2), and L^{F} may denote a loss calculated according to Equation (1).

For convenience of description, FIG. 3 illustrates an example of predicting gene expression for one of a plurality of spot images in the histology image 310, but the disclosure is not limited thereto. For example, the electronic device may predict gene expression for each spot image by performing one or more of the operations described above, on each of a plurality of spot images as a target spot image.

For example, the global encoder 320 may dependently receive, as input, data associated with a plurality of spot images, and output global tokens corresponding to the plurality of spot images, and the global tokens corresponding to the respective spot images may be used to predict gene expression for the respective spot images. For example, the local encoder 330 may independently receive, as input, each spot image and output a local token corresponding to the spot image, and the local token corresponding to each spot image may be used to predict gene expression for the spot image. For example, the neighbor encoder 340 may independently receive, as input, a neighbor image including each spot image and output a neighbor token corresponding to the spot image, and the neighbor token corresponding to each spot image may be used to predict gene expression for the spot image.

FIG. 4 is a diagram illustrating an image region used as the basis for an embedding model to embed a target spot image, according to an embodiment.

In describing FIG. 4, redundant descriptions provided above with reference to any one of FIGS. 1A to 3 may be omitted.

Referring to FIG. 4, a histology image 400 may include a plurality of spot images. In an embodiment, the histology image 400 may be segmented into a plurality of spot images having a predefined size. For example, the histology image 400 may be segmented into spot images having a size of 224×224, based on predefined center coordinates. For example, the spot images may not be completely adjacent to or overlap each other. For example, as illustrated in FIG. 4, the alignment and spacing between the spot images in the histology image 400 may not be uniform, and some spacing (gaps or regions) may exist between spot images included in the histology image 400. Thus, the regions of all spot images in the histology image 400 may not cover the entire region of the histology image 400.

In an embodiment, the electronic device may obtain global feature data corresponding to a target spot image 410, based on a plurality of spot images including the target spot image 410 by using the global embedding model. For example, an image region that is used as the basis for the global embedding model to embed or encode the target spot image 410 in the histology image 400 may include a plurality of spot images. For example, the global embedding model may extract global feature data regarding the target spot image 410 based on image information e.g., biological information or contextual information) included within a range of a plurality of spot images and/or spatial position information about the spot images.

In an embodiment, the histology image 400 or the spot images may not be directly input into the global embedding model. For example, the electronic device may extract, from spot images, initial feature data corresponding to each spot image by using a pre-trained model, and use the initial feature data corresponding to each spot image as input data for the global embedding model. For example, the electronic device may obtain global feature data (e.g., global tokens) corresponding to each spot image that is output from the global embedding model, by inputting the initial feature data corresponding to each spot image into the global embedding model.

In an embodiment, the electronic device may obtain local feature data corresponding to the target spot image 410, based on the target spot image 410 by using the local embedding model. For example, an image region that is used as the basis for the local embedding model to embed or encode the target spot image 410 in the histology image 400 may be the target spot image 410. For example, the local embedding model may extract local feature data regarding the target spot image 410 based on image information (e.g., biological information or contextual information) included within a range of the target spot image 410.

In an embodiment, the local embedding model may receive the target spot image 410 (or data associated with the target spot image 410) as individual input data. For example, the electronic device may input the target spot image 410 into the local embedding model independently of other spot images, to obtain local feature data (e.g., local tokens) corresponding to the target spot image 410. For example, before inputting each spot image into the local embedding model, the electronic device may perform a normalization process to adjust the pixel values of each spot image to fall within the range of 0 to 1.

In an embodiment, the electronic device may obtain neighbor feature data corresponding to the target spot image 410 based on a neighbor image 420 including the target spot image 410, by using the neighbor embedding model. For example, an image region that is used as the basis for the neighbor embedding model to embed or encode the target spot image 410 in the histology image 400 may be the neighbor image 420. For example, the neighbor embedding model may extract neighbor feature data regarding the target spot image 410 based on image information (e.g., biological information or contextual information) included within a range of the neighbor image 420.

The neighbor image 420 may be an image of a partial region in the histology image 400 that includes the target spot image 410. For example, the neighbor image 420 may be an image having a predetermined (predefined or preset) size (e.g., a size of 1120×1120) and further including regions surrounding the target spot image 410. As illustrated in FIG. 4, the neighbor image 420 may be an image that includes surrounding regions directly adjacent to the target spot image 410, rather than being composed of a group of spot images adjacent to the target spot image 410. Accordingly, even when the alignment and spacing between the spot images are not uniform, the neighbor image 420 may include direct neighbor regions of the target spot image 410, and may include regions that are not included in the spot images in the histology image 400 (e.g., spatial spacings between the spot images). Thus, the neighbor embedding model may output (extract, generate, or determine) neighbor feature data corresponding to the target spot image 410 by considering biological information about some surrounding regions that are not included in the target spot image 410 or surrounding spot images.

In an embodiment, when the target spot image 410 is a spot image located at an edge of the histology image 400, the electronic device may obtain the neighbor image 420 having a predetermined (predefined or preset) size or resolution by using a zero padding technique. For example, when the target spot image 410 is located at an edge of the histology image 400, the electronic device may obtain a neighbor image having a predetermined size or resolution and centered on the target spot image 410, with zero data filled in surrounding regions outside the histology image 400.

In an embodiment, the neighbor image 420 may be segmented into nₙₑ (e.g., 25) sub-regions (nₙₑ is a natural number) having a predetermined size (e.g., 224×224). For example, the sub-regions of the neighbor image 420 may not overlap each other, and there may be no spatial spacing between the sub-regions. For example, the sub-regions may be directly adjacent to each other, and the alignment between the sub-regions may be uniform. Thus, the sub-regions of the neighbor image 420, and regions of other spot images around the target spot image 410 may not coincide with each other. For example, 25 sub-regions of the neighbor image 420 may not coincide with other 25 spot images closest to the target spot image 410.

In an embodiment, the neighbor image 420 or the sub-regions may not be directly input into the neighbor embedding model. For example, the electronic device may extract, from the sub-regions, initial feature data regarding each sub-region by using a pre-trained model, and use the initial feature data regarding each sub-region as input data for the neighbor embedding model. For example, the electronic device may obtain neighbor feature data corresponding to the target spot image 410 that is output from the neighbor embedding model, by inputting the initial feature data corresponding to each sub-region into the neighbor embedding model.

FIG. 5 is a diagram illustrating an example of obtaining fusion feature data based on global feature data, local feature data, and neighbor feature data, which correspond to a target spot image, according to an embodiment.

In describing FIG. 5, redundant descriptions provided above with reference to any one of FIGS. 1A to 4 may be omitted.

The electronic device may predict gene expression for a target spot image based on global feature data, local feature data, and neighbor feature data that correspond to the target spot image. In an embodiment, a method, performed by an electronic device, of efficiently integrating various types of feature data with minimal additional cost, for gene expression prediction may be provided. For example, the electronic device may obtain fusion feature data corresponding to the target spot image based on global feature data, local feature data, and neighbor feature data that correspond to the target spot image, by using the feature fusion model, and predict gene expression for the target spot image based on the obtained fusion feature data.

In an embodiment, information exchange between neighbor feature data (e.g., neighbor tokens) and local feature data (e.g., local tokens) may not be performed directly, but may be performed through global feature data (e.g., global tokens). For example, the electronic device may use the feature fusion model to obtain global-local fusion feature data corresponding to the target spot image based on the global feature data and the local feature data, and to obtain global-neighbor fusion feature data corresponding to the target spot image based on the global feature data and the neighbor feature data. For example, the fusion feature data corresponding to the target spot image may include the global-local fusion feature data and the global-neighbor fusion feature data both corresponding to the target spot image.

In an embodiment, the electronic device may predict gene expression for the target spot image based on the global-local fusion feature data and the global-neighbor fusion feature data by using a prediction model. For example, the electronic device may obtain final fusion feature data corresponding to the target spot image based on the global-local fusion feature data and the global-neighbor fusion feature data, and predict gene expression for the target spot image based on the final fusion feature data by using the prediction model. For example, the electronic device may obtain fusion feature data (e.g., a fusion token) corresponding to the target spot image based on Equation (4) below.

$${z}_{i}^{GT} = CrossAttn ( {z}_{i}^{GI} , {z}_{i}^{Ta} ) ∈ {R}^{d} \\ {z}_{i}^{GN} = CrossAttn ( {z}_{i}^{GI} , {z}_{i}^{Ne} ) ∈ {R}^{d} \\ {z}_{i}^{GTN} = Sum ( {z}_{i}^{GT} , {z}_{i}^{GN} ) ∈ {R}^{d} \qquad ( 4 )$$

In Equation (4), zᵢ^{GI} may denote a global token corresponding to the i-th spot image, zᵢ^{Ta} may denote a local token corresponding to the i-th spot image, and zᵢ^{Ne} may denote a neighbor token corresponding to the i-th spot image. The operation CrossAttn(x,y) may denote a cross-attention operation for y with respect to x, and the operation Sum(x,y) may denote a sum operation of x and y. For example, the operation CrossAttn(x,y) may denote a dot-product attention operation that uses x as a query Q, and uses a key (K)-value (V) pair formed by y. zᵢ^{GT} may denote a global-local fusion token corresponding to the i-th spot image, and zᵢ^{GN} may denote a global-neighbor fusion token corresponding to the i-th spot image. zᵢ^{GTN} may denote a final fusion token used to infer a gene expression level in the i-th spot image.

Referring to FIG. 5, the feature fusion model may include one or more attention layers (models or blocks) 510 and 520. For example, the feature fusion model may include a first attention layer 510 configured to fuse the global token 326 with the neighbor tokens 344, and a second attention layer 520 configured to fuse the global token 326 with the local tokens 336. For effective information exchange between different types of tokens, and improved contextual understanding, the electronic device may perform cross-attention operations of the attention layers of the feature fusion model.

As illustrated in FIG. 5, the electronic device may obtain a global-neighbor fusion token 512 corresponding to the target spot image by performing a dot-product attention operation of the first attention layer 510 by using a key-value pair based on the neighbor tokens 344, and a query based on the global token 326. For example, the first attention layer 510 may output the global-neighbor fusion token 512 that reflects the relative importance (or similarity) of each neighbor token with respect to the global token 326. In addition, the electronic device may obtain a global-local fusion token 522 corresponding to the target spot image by performing a dot-product attention operation of the second attention layer 520 by using a key-value pair based on the local tokens 336, and a query based on the global token 326. For example, the second attention layer 520 may output the global-local fusion token 522 that reflects the relative importance (or similarity) of each local token with respect to the global token 326.

Referring to FIG. 5, the electronic device may obtain (calculate or determine) the final fusion token 352 corresponding to the target spot image by summing the global-neighbor fusion token 512 and the global-local fusion token 522. For example, the electronic device may obtain the final fusion token 352 corresponding to the target spot image by performing an element-wise sum or an element-wise weighted sum of the global-neighbor fusion token 512 and the global-local fusion token 522. The electronic device may predict gene expression for the target spot image based on the final fusion token 352 by using the prediction model. For example, the electronic device may obtain a gene expression prediction value (or level) for the target spot image that is output from the prediction model, by inputting the final fusion token 352 into the prediction model.

FIG. 6 is a diagram illustrating an example of obtaining global feature data corresponding to a target spot image based on initial feature data corresponding to a plurality of spot images, according to an embodiment.

In describing FIG. 6, redundant descriptions provided above with reference to any one of FIGS. 1A to 5 may be omitted.

In an embodiment, the electronic device may obtain global feature data corresponding to a target spot image, based on a plurality of spot images in a histology image by using the global embedding model. For example, the electronic device may obtain global feature data corresponding to a plurality of spot images that is output from the global embedding model, by inputting initial feature data corresponding to the plurality of spot images into the global embedding model. For example, the electronic device may obtain global feature data corresponding to the target spot image that is extracted based on image information (e.g., biological information or contextual information within the image) and spatial positional information about a plurality of spot images.

Referring to FIG. 6, the global embedding model may include one or more transformer models (layers or models) as sub-models. For example, the global embedding model may include a ViT model configured to learn long-term dependencies through an attention operation (e.g., a self-attention operation). For example, the one or more transformer models of the global embedding model may encode (or embed) global contextual information about spot images in a histology image, based on correlations between the spot images. For example, the electronic device may perform an attention operation on tokens corresponding to a plurality of spot images according to the transformer models.

Referring to FIG. 6, the global embedding model may include one or more positional information encoding models (blocks, layers, or generators) 620 as sub-models. In an embodiment, the electronic device may obtain global feature data corresponding to a target spot image in a histology image, wherein positional information or spatial information about the target spot image is encoded (embedded or reflected) in the global feature data, by using the one or more positional information encoding models of the global embedding model. For example, the one or more positional information encoding models of the global embedding model may encode (or embed) spatial information or positional information about each spot image in the histology image.

FIG. 6 may show an example of a structure of a global embedding model according to an embodiment. Referring to FIG. 6, the global embedding model may include a first transformer model 610, a positional information encoding model 620, and (L−1) transformer models 630. For example, the electronic device may obtain final global feature data corresponding to the plurality of spot images by updating the global feature data based on results of a series of operations (or sub-models) of the global embedding model.

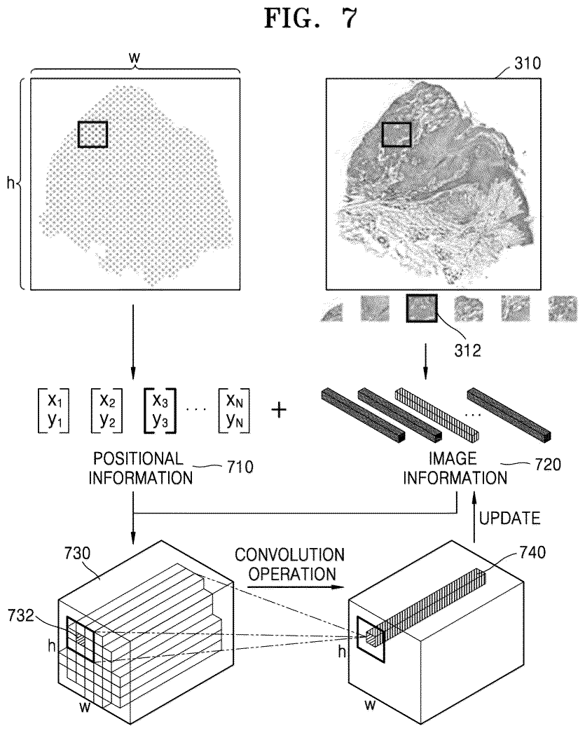

For example, according to the structure of the global embedding model, the electronic device may perform an operation of the first transformer model 610 on initial feature tokens 324 corresponding to the plurality of spot images, by inputting the initial feature tokens 324 corresponding to the plurality of spot images into the first transformer model 610. The electronic device may obtain, as global tokens corresponding to the plurality of spot images, first intermediate tokens output by the first transformer model 610. The electronic device may perform an operation of the positional information encoding model 620 to encode spatial positional information about the spot images, by inputting the first intermediate tokens into the positional information encoding model 620. An example of a positional encoding operation of the positional information encoding model 620 in an embodiment may be described below with reference to FIG. 7, and redundant descriptions may be omitted.

The electronic device may obtain second intermediate tokens output by the positional information encoding model 620, and update the global tokens by adding the second intermediate tokens to the first intermediate tokens. For example, the global tokens may be updated, from the first intermediate tokens to the sum of the first intermediate tokens and the second intermediate tokens. For example, the electronic device may perform an element-wise sum or an element-wise weighted sum of the first intermediate tokens and the second intermediate tokens.

For example, according to the structure of the global embedding model, the electronic device may sequentially perform a series of transformer model operations (e.g., self-attention operations) by inputting the updated global tokens into the (L−1) transformer models 630. For example, the electronic device may update the global tokens by performing an attention operation on the global tokens multiple times. For example, the electronic device may obtain final global tokens 640 by updating the global tokens with tokens obtained through the last operation of the global embedding model.

Each of the plurality of global tokens 640 may correspond to one spot image, and the global token to be used from among the plurality of global tokens 640 may vary depending on the spot image for which gene expression is to be predicted. For example, the electronic device may identify (select or determine) the global token 326 corresponding to the target spot image from among the plurality of global tokens 640. The global token 326 corresponding to the target spot image from among the plurality of global tokens 640 may be used for gene expression prediction for the target spot image.

FIG. 6 illustrates that tokens output from the first transformer model 610 of the global embedding model are input into the positional information encoding model 620, but the disclosure is not limited thereto. For example, tokens output from the e-th transformer block (e is a natural number of 1 or greater but less than L) of the global embedding model may be input into the positional information encoding model 620. In this case, the electronic device may obtain global feature tokens 640 corresponding to the plurality of spot images based on tokens output from the positional information encoding model 620, and tokens output from the e-th transformer model. For example, the electronic device may obtain the global feature tokens 640 corresponding to the plurality of spot images by summing (e.g., element-wise sum or weighted sum) the tokens output from the positional information encoding model 620, and the tokens output from the e-th transformer model, and inputting the sum into an (e+1)-th encoder.

FIG. 7 is a diagram illustrating an example of encoding positional information about a spot image in a histology image, according to an embodiment.

In describing FIG. 7, redundant descriptions provided above with reference to any one of FIGS. 1A to 6 may be omitted.

Tissue slices from a biopsy (i.e., histology images and WSIs) have the characteristic that an image region may not have a quadrangular shape but an irregular shape, unlike general images, and thus, the positional information about a spot image in the histology image 310 may not be properly encoded when using related-art positional information encoding methods. To overcome the limitations of related-art positional information encoding methods designed for regular square-shaped images or images having a fixed size, in an embodiment, the electronic device may use a positional information encoding model (block or layer) specifically designed for a high-resolution histology image 310. For example, to encode positional information 710 about a spot image, the electronic device may use an atypical positional encoding model that is modified to be suitable for the histology image 310 having an irregular image shape or a variable image size. For example, the atypical positional encoding model may encode absolute position information about a plurality of spot images in the histology image 310.

FIG. 7 may illustrate an example of an operation by the positional information encoding model 620 of FIG. 6. In an embodiment, the electronic device may obtain global feature data in which image information 720 (e.g., biological information or contextual information included in the image) and the positional information 710 (or spatial information) about spot images are reflected (encoded or embedded), by performing an operation of the global embedding model including the positional information encoding model. For example, the electronic device may obtain first intermediate tokens in which the image information 720 about the spot images is reflected (or embedded), by performing an operation of the first transformer model (or the e-th transformer model) of the global embedding model, and obtain second intermediate tokens in which the positional information 710 about the spot images is reflected (or encoded), by performing an operation of the positional information encoding model on the first intermediate tokens. For example, the electronic device may update global tokens corresponding to the spot images, from the first intermediate tokens to the sum of the first intermediate tokens and the second intermediate tokens.

Referring to FIG. 7, first intermediate tokens corresponding to n spot images may include n d-dimensional tokens. The electronic device may reshape the first intermediate tokens in a space [symbol] ^{n×d} into data in a space [symbol] ^{h×w×d} 730, according to the positional information 710 about the spot images. For example, the electronic device may restore the original positional information 710 about the spot images by arranging the first intermediate tokens in the space [symbol] ^{n×d}, in the space [symbol] ^{h×w×d} 730, according to the positional information 710 about the spot images. h and w in the space [symbol] ^{h×w×d} 730 may represent the maximum values of the x-coordinate and the y-coordinate of the spot images in the histology image 310, respectively. Data regarding regions in the space [symbol] ^{h×w×d} 730 that do not correspond to the spot images may be temporarily filled with 0.

In an embodiment, the electronic device may perform a zero-padding convolution operation of the positional information encoding model, on the data in the space [symbol] ^{h×w×d} 730. For example, the electronic device may obtain second intermediate tokens corresponding to the spot images by performing a convolution operation on the data in the space [symbol] ^{h×w×d} 730, and filling again data regarding regions in the space [symbol] ^{h×w×d} 730 that do not correspond to spot images, with 0. The electronic device may revert the second intermediate tokens in the space [symbol] ^{h×w×d} 730 back to the data format in the space [symbol] ^{n×d}.

In an embodiment, the electronic device may update the global tokens corresponding to the spot images by reflecting a result of a positional information encoding operation. For example, the electronic device may update the global tokens corresponding to the spot images, based on the second intermediate tokens in the space [symbol] ^{n×d}. For example, the electronic device may update the global tokens, from the first intermediate tokens to a sum (e.g., an element-wise sum or an element-wise weighted sum) of the first intermediate tokens and the second intermediate tokens. For example, the electronic device may update the global token corresponding to the target spot image 312, with a sum of a first intermediate token 732 and a second intermediate token 740 that correspond to the target spot image 312.

When encoding the positional information 710 by using the atypical positional encoding model according to an embodiment, the positional information 710 about the spot images may be dynamically encoded even when the size or shape of the histology image 310 is variable. The electronic device may obtain final global tokens corresponding to the plurality of spot images by performing subsequent operations (e.g., remaining transformer encoder operations of the global embedding model) based on the updated global tokens.

One or more of the above-described embodiments of the global embedding model in FIG. 6 or 7 may also be applied to the neighbor embedding model. For example, the neighbor embedding model may be configured in a structure identical or similar to the structure of the global embedding model of FIG. 6. For example, the neighbor embedding model may include one or more transformer models and positional information encoding models. For example, the processes or operations performed on the plurality of spot images in one or more of the embodiments described above for the global embedding model may be performed on a plurality of sub-regions of a neighbor image. For example, the processes or operation performed on the target spot image in one or more of the embodiments described above for the global embedding model may be performed on a target spot image as a neighbor region of a neighbor image.

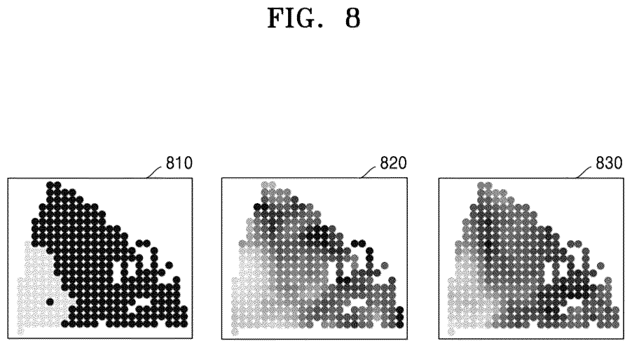

FIG. 8 is a diagram illustrating the performance of a gene expression prediction model according to an embodiment.

In describing FIG. 8, redundant descriptions provided above with reference to any one of FIGS. 1A to 7 may be omitted.

The expression level of a particular gene may be used as an important biomarker to predict and evaluate the occurrence, progression, treatment response, and the like for cancer. For example, the CLDN4 gene and GNAS gene may be used as markers for breast cancer. For example, the expression level of a particular gene is high, it may be predicted or estimated that breast cancer has occurred, is occurring, or is expected to occur. According to an embodiment, the electronic device may predict, from a histology image, the expression level of a particular gene on a spot-by-spot basis, and because the expression level of a particular gene may be utilized as a biomarker, a result of gene expression prediction according to an embodiment may be utilized to predict and evaluate the occurrence, progression, treatment response, and the like for cancer.

FIG. 8 may show a tumor region image 810 in a histology image, a ground-truth GNAS gene expression value image 820, and a GNAS gene expression prediction value image 830. The tumor region image 810 of FIG. 8 represents a tumor region image annotated by a pathologist on a spot-by-spot basis, and the GNAS gene expression prediction value image 830 may represent a GNAS gene expression value on a spot-by-spot basis predicted by a gene expression prediction model (e.g., the artificial intelligence model 300 of FIG. 3) according to an embodiment. In the ground-truth GNAS gene expression value image 820 and the GNAS gene expression prediction value image 830, the brightness of each spot may represent a gene expression value, and as the brightness increases, the expression value for the GNAS gene may decrease. In the tumor region image 810, dark spots may represent tumor regions, and bright spots may represent normal regions.

As illustrated in FIG. 8, by comparing the ground-truth GNAS gene expression value image 820 with the GNAS gene expression prediction value image 830, it may be confirmed that the GNAS gene expression values predicted according to an embodiment are similar to the ground-truth GNAS gene expression values, throughout the histology image. In detail, in both the ground-truth GNAS gene expression value image 820 and the GNAS gene expression prediction value image 830, the brightnesses of spots in the lower left region are high, which may indicate low expression levels of the GNAS gene. In addition, in both the ground-truth GNAS gene expression value image 820 and the GNAS gene expression prediction value image 830, the brightnesses of spots in the right region are low, which may indicate high expression levels of the GNAS gene.

In addition, as illustrated in FIG. 8, it may be confirmed that the GNAS gene expression level is predicted to be low in the lower left region of the GNAS gene expression prediction value image 830 corresponding to the region annotated as a normal spot in the tumor region image 810, and the GNAS gene expression level is predicted to be high in the right region of the GNAS gene expression prediction value image 830 corresponding to the region annotated as a tumor spot in the tumor region image 810. Therefore, the method of predicting gene expression according to an embodiment may achieve improved prediction performance, thereby activating the function of a gene as a biomarker, and further contributing to the development of pathological fields such as cancer prediction, diagnosis, or treatment.

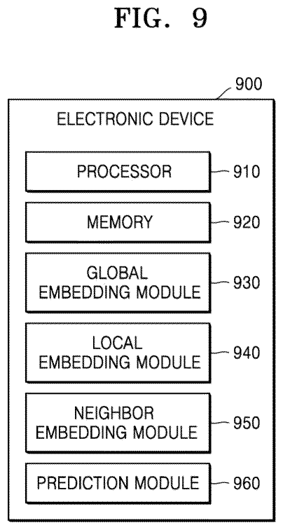

FIG. 9 is a block diagram illustrating an example of an electronic device according to an embodiment.

In describing FIG. 9, redundant descriptions provided above with reference to any one of FIGS. 1A to 8 may be omitted.

An electronic device 900 illustrated in FIG. 10 9 a device for predicting spot-level gene expression from a histology image by using an artificial intelligence model, and may be a user terminal or a server device. For example, the electronic device 900 may be a user terminal such as a desktop computer, a laptop computer, a notebook, a smart phone, a tablet PC, a mobile phone, a smart watch, a wearable device, an augmented reality (AR) device, or a virtual reality (VR) device.

In an embodiment, an electronic device for training or updating an artificial intelligence model to predict spot-level gene expression from a histology image may be a user terminal or a server device that is the same as or different from the electronic device 900 for performing prediction or inference by using an artificial intelligence model. For example, in a case in which the electronic device for training or updating an artificial intelligence model and the electronic device 900 (i.e., the electronic device for performing a prediction or inference operation) are different from each other, the electronic device 900 may receive a trained or updated artificial intelligence model from the electronic device for training or updating an artificial intelligence model. One or more embodiments to be described below regarding the electronic device 900 may also be applied to the electronic device for training or updating an artificial intelligence model to predict spot-level gene expression from a histology image.

In an embodiment, the artificial intelligence model may be dynamically updated as a prediction or inference operation is performed. For example, at least some weights of the artificial intelligence model or at least one sub-model of the artificial intelligence model may be dynamically updated as a prediction or inference operation is performed. For example, in a case in which the electronic device for updating an artificial intelligence model and the electronic device 900 are the same as each other, the electronic device 900 may perform prediction or inference by using an artificial intelligence model and update the artificial intelligence model at the same time. For example, in a case in which the electronic device for updating an artificial intelligence model and the electronic device 900 are different from each other, the electronic device for updating an artificial intelligence model may perform prediction or inference by using an artificial intelligence model, receive identified, generated, or calculated data, and update the artificial intelligence model based on the received data.

In an embodiment of the disclosure, the electronic device 900 may include, but is not limited to, at least one processor 910 and a memory 920. The processor 910 may be electrically connected to the components included in the electronic device 900 to perform computation or data processing for control and/or communication of the components included in the electronic device 900. In an embodiment, the processor 910 may load, into the memory, a request, a command, or data received from at least one of other components, process the request, command, or data, and store process result data in the memory. For example, the processor 910 may perform control to process data according to predefined operation rules, algorithms, or an artificial intelligence model (e.g., a neural network model) stored in the memory 920. The processor 910 may include at least one of general-purpose processors such as a CPU, an AP, or a DSP, dedicated graphics processors such as a GPU or a vision processing unit (VPU), or dedicated artificial intelligence processors such as an NPU. For example, in a case in which the processor 910 is a dedicated artificial intelligence processor, the dedicated artificial intelligence processor may be designed with a hardware structure specialized for processing a particular artificial intelligence model.

The processor 910 may include various processing circuits and/or a plurality of processors. For example, the term ‘processor’ as used herein, including in the claims, may include various processing circuitries including at least one processor. One or more of the at least one processor may be configured to perform one or more of the functions described herein, individually and/or collectively in a distributed manner. In the disclosure, when a‘processor’, ‘at least one processor’, or ‘one or more processors’ are described as being configured to perform a plurality of functions, this may include a situation where one processor performs some of the functions and the other processor(s) performs the other functions, and a situation where a single processor performs all of the functions. In addition, the at least one processor may include a combination of processors configured to perform various functions in a distributed manner. The at least one processor may execute program instructions to achieve or perform various functions.

The memory 920 may be electrically connected to the processor 910, and may store one or more modules, artificial intelligence models, programs, instructions, or data related to the operations of the components included in the electronic device 900. For example, the memory 920 may store one or more modules, artificial intelligence models, programs, instructions, or data for the processor 910 to perform processing, computation, and control. The memory 920 may include at least one of a flash memory-type storage medium, a hard disk-type storage medium, a multimedia card micro-type storage medium, card-type memory (e.g., SD or XD memory), random-access memory (RAM), static RAM (SRAM), read-only memory (ROM), electrically erasable programmable ROM (EEPROM), programmable ROM (PROM), magnetic memory, a magnetic disc, and an optical disc.

In an embodiment, the memory 920 may store data or information that is received or generated by the electronic device 900, or is input into the electronic device 900. For example, the memory 920 may store a received histology image, weights of an artificial intelligence model, and the like. For example, the memory 920 may store a gene expression prediction result for a histology image (or a spot image). For example, the memory 920 may store data or information that is received or generated by the electronic device 900, or is input into the electronic device 900, in a compressed form.

A software module or model included in the memory 920 may be executed under control or command of the processor 910. The software module or model may include operations, programs, or algorithms for deriving output data for input data. In an embodiment, the memory 920 may include at least one neural network model, an artificial intelligence model, a machine learning model, a statistical model, an algorithm, and the like for image processing. For example, the memory 920 may include an artificial intelligence model trained to predict or infer spot-level gene expression from a histology image. For example, the memory 920 may include values (weights) of a plurality of parameters that constitute an artificial intelligence model.

The predefined operation rules, algorithms, or artificial intelligence model included in the memory 920 may have been generated via a training process. Here, being generated via a training process may mean that predefined operation rules or artificial intelligence model set to perform desired characteristics (or purposes), is generated by training a basic artificial intelligence model by using a learning algorithm that utilizes a large amount of training data. The training process may be performed by a device itself on which artificial intelligence according to the disclosure is performed, or by a separate server and/or system. Examples of learning algorithms may include, for example, supervised learning, unsupervised learning, semi-supervised learning, and reinforcement learning, but are not limited thereto.

The artificial intelligence model may include a plurality of neural network layers. Each of the neural network layers may have a plurality of weight values, and may perform a neural network arithmetic operation via an arithmetic operation between an arithmetic operation result of a previous layer and the plurality of weight values. The plurality of weight values in each of the plurality of neural network layers may be optimized as a result of training the artificial intelligence model. For example, the plurality of weight values may be updated to reduce or minimize a loss or cost value obtained by the artificial intelligence model during a training process. The artificial intelligence model may include a deep neural network (DNN), and may be, for example, a CNN, a long short-term memory (LSTM), a DNN, a recurrent neural network (RNN), a restricted Boltzmann machine (RBM), a deep belief network (DBN), a bidirectional recurrent deep neural network (BRDNN), a transformer, a deep Q-network, or the like, but is not limited thereto. The artificial intelligence model may include a statistical method model, for example, logistic regression, a Gaussian Mixture Model (GMM), a support vector machine (SVM), latent Dirichlet allocation (LDA), or decision tree, but is not limited thereto.

In an embodiment, the artificial intelligence model may include one or more sub-models. For example, the artificial intelligence model may include sub-models distinguishable from each other based on function, input data, output data, loss function, or structure. For example, the artificial intelligence model may include a plurality of sub-models that are arbitrarily or logically distinguishable from each other.

In an embodiment, at least one sub-model included in the artificial intelligence model may be trained or updated together with other sub-models. For example, as the artificial intelligence model is trained or updated, at least some of the sub-models included in the artificial intelligence model may be dynamically trained or updated. In an embodiment, at least one sub-model included in the artificial intelligence model may not be trained or updated even when other sub-models are trained or updated. For example, at least one sub-model may have completely trained, and may maintain fixed weights without being updated even when the artificial intelligence model is trained or updated.

In an embodiment, a model for predicting spot-level expression from a histology image may include, as sub-models, at least some of a global embedding model, a local embedding model, a neighbor embedding model, a fusion model, or a prediction model. In an embodiment, the global embedding model may include one or more transformer models (or layers) and positional information encoding models (or layers), as sub-models. In an embodiment, the neighbor embedding model may include one or more transformer models (or layers) and positional information encoding models (or layers), as sub-models.

Some modules that perform at least one operation of the electronic device 900 may be implemented as hardware modules, software modules, and/or a combination thereof. The memory 920 may include a software module that performs at least some of the operations of the electronic device 900 described above. In an embodiment, the software module included in the memory 920 may be executed by the processor 910 to perform an operation. For example, the processor 910 may perform operations of the software module included in the memory 920. The artificial intelligence model included in the memory 920 may be included in the software module, may be executed by the software module, or may be the software module itself. At least some of the modules of the electronic device 900 may include a plurality of sub-modules or may constitute one module.

Referring to FIG. 9, the electronic device 900 may include, but is not limited to, a global embedding module 930, a local embedding module 940, a neighbor embedding module 950, and a prediction module 960. For example, the electronic device 900 may further include a feature fusion module. For example, the electronic device 900 may include some of the global embedding module 930, the local embedding module 940, the neighbor embedding module 950, or the prediction module 960. In this case, the other modules may be included in another electronic device, and the electronic device 900 and the other electronic device may be connected to each other or communicate with each other in a wired or wireless manner to constitute a system.

FIG. 9 illustrates that the global embedding module 930, the local embedding module 940, the neighbor embedding module 950, and the prediction module 960 are separate components from the processor 910 and the memory 920, but the disclosure is not limited thereto. For example, at least one of the global embedding module 930, the local embedding module 940, the neighbor embedding module 950, and the prediction module 960 may be included in the processor 910 as a hardware module, or may be a separate hardware model. For example, at least one of the global embedding module 930, the local embedding module 940, the neighbor embedding module 950, and the prediction module 960 may be included in the memory 920 as a software module. For example, a software component of each module may be included in memory 920.

In an embodiment, the global embedding module 930 may include a model (e.g., a global feature extraction model or a global embedding model) configured to extract spot-level global feature data from a histology image. In an embodiment, the local embedding module 940 may include a model (e.g., a local feature extraction model or a local embedding model) configured to extract spot-level local feature data from a histology image. In an embodiment, the neighbor embedding module 950 may include a model (e.g., a neighbor feature extraction model or a neighbor embedding model) configured to extract spot-level neighbor feature data from a histology image. In an embodiment, the prediction module 960 may include a model (e.g., a prediction model) configured to predict spot-level gene expression based on global feature data, local feature data, and neighbor feature data, in units of spots of a histology image.

The electronic device 900 may include more components than those illustrated in FIG. 9. For example, the electronic device 900 may further include a communication interface (or a communication module) for communication with an external device. For example, the electronic device 900 may further include an input/output device and/or an input/output interface. For example, the electronic device 900 may further include a camera sensor for generating a histology image.

In the disclosure, redundant descriptions provided above with reference to FIGS. 1A to 9 may have been omitted, and one or more embodiments described above with reference to FIGS. 1A to 9 may be applied or implemented in combination with each other.

In the disclosure, an operation described as being performed by an electronic device may be performed or executed by a module included or stored in the electronic device, may be performed or executed by at least one processor of the electronic device, or may be performed by at least one processor of the electronic device controlling a module included or stored in the electronic device. In the disclosure, operations described as being performed by a module, a model or an encoder may be performed by an electronic device (or at least one processor of the electronic device) including the module, model or encoder. In the disclosure, operations described as being performed by an electronic device by using a module, a model, or an encoder may be performed by the module, the model, or the encoder.

In an embodiment, a method, performed by an electronic device, of predicting gene expression from a histology image by using an artificial intelligence model may include obtaining, based on a first spot image and a second spot image in the histology image, global feature data corresponding to the first spot image by using a first artificial intelligence model. In an embodiment, the method may include obtaining, from the first spot image, local feature data corresponding to the first spot image by using a second artificial intelligence model. In an embodiment, the method may include obtaining, by using a third artificial intelligence model, neighbor feature data corresponding to the first spot image based on a neighbor image including the first spot image and a surrounding region in the histology image. In an embodiment, the method may include predicting gene expression for the first spot image based on the global feature data, the local feature data, and the neighbor feature data by using a fourth artificial intelligence model.

In an embodiment, the predicting of the gene expression for the first spot image based on the global feature data, the local feature data, and the neighbor feature data by using the fourth artificial intelligence model may include obtaining fusion feature data corresponding to the first spot image based on the global feature data, the local feature data, and the neighbor feature data by using a fifth artificial intelligence model. In an embodiment, the predicting of the gene expression for the first spot image based on the global feature data, the local feature data, and the neighbor feature data by using the fourth artificial intelligence model may include predicting the gene expression for the first spot image based on the fusion feature data by using the fourth artificial intelligence model.

In an embodiment, the obtaining of the fusion feature data corresponding to the first spot image may include obtaining, by using the fifth artificial intelligence model, global-neighbor fusion feature data corresponding to the first spot image based on the global feature data and the neighbor feature data, and obtaining, by using the fifth artificial intelligence model, global-local fusion feature data corresponding to the first spot image based on the global feature data and the local feature data. In an embodiment, the obtaining of the fusion feature data corresponding to the first spot image may include obtaining the fusion feature data corresponding to the first spot image based on the global-neighbor fusion feature data and the global-local fusion feature data.

In an embodiment, the obtaining of the neighbor feature data may include segmenting the neighbor image into a plurality of sub-regions including a first sub-region and a second sub-region. In an embodiment, the obtaining of the neighbor feature data may include obtaining, from the first sub-region by using a trained model, first initial feature data corresponding to the first sub-region, and obtaining, from the second sub-region by using the trained model, second initial feature data corresponding to the second sub-region. In an embodiment, the obtaining of the neighbor feature data may include obtaining the neighbor feature data corresponding to the first spot image based on the first initial feature data and the second initial feature data by using the third artificial intelligence model.

In an embodiment, the obtaining of the global feature data may include obtaining, by using a trained model, third initial feature data from the first spot image, and obtaining, by using the trained model, fourth initial feature data from the second spot image. In an embodiment, the obtaining of the global feature data may include obtaining the global feature data corresponding to the first spot image based on the third initial feature data and the fourth initial feature data by using the first artificial intelligence model.

In an embodiment, the first artificial intelligence model may include a positional information encoding model configured to encode positional information in the histology image.

In an embodiment, the obtaining of the global feature data may include obtaining the global feature data corresponding to the first spot image, wherein positional information about the first spot image is encoded in the global feature data, by using the positional information encoding model.

In an embodiment, there may be provided a computer-readable recording medium having recorded thereon a program for executing, on a computer, the method of predicting gene expression from a histology image by using an artificial intelligence model.

In an embodiment, an electronic device for predicting gene expression from a histology image by using an artificial intelligence model may include a memory storing one or more instructions, and at least one processor configured to execute the one or more instructions. In an embodiment, the at least one processor may be configured to execute the one or more instructions stored in the memory to cause the electronic device to obtain, based on a first spot image and a second spot image in the histology image, global feature data corresponding to the first spot image by using a first artificial intelligence model. In an embodiment, the at least one processor may be further configured to execute the one or more instructions stored in the memory to cause the electronic device to obtain, from the first spot image, local feature data corresponding to the first spot image by using a second artificial intelligence model. In an embodiment, the at least one processor may be further configured to execute the one or more instructions stored in the memory to cause the electronic device to obtain, by using a third artificial intelligence model, neighbor feature data corresponding to the first spot image based on a neighbor image including the first spot image and a surrounding region in the histology image. In an embodiment, the at least one processor may be further configured to execute the one or more instructions stored in the memory to cause the electronic device to predict gene expression for the first spot image based on the global feature data, the local feature, data and the neighbor feature data by using a fourth artificial intelligence model.

In an embodiment, the at least one processor may be further configured to execute the one or more instructions to cause the electronic device to obtain fusion feature data corresponding to the first spot image based on the global feature data, the local feature data, and the neighbor feature data by using a fifth artificial intelligence model. In an embodiment, the at least one processor may be further configured to execute the one or more instructions to cause the electronic device to predict the gene expression for the first spot image based on the fusion feature data by using the fourth artificial intelligence model.

In an embodiment, the at least one processor may be further configured to execute the one or more instructions to cause the electronic device to obtain, by using the fifth artificial intelligence model, global-neighbor fusion feature data corresponding to the first spot image based on the global feature data and the neighbor feature data, and obtain, by using the fifth artificial intelligence model, global-local fusion feature data corresponding to the first spot image based on the global feature data and the local feature data. In an embodiment, the at least one processor may be further configured to execute the one or more instructions to cause the electronic device to obtain the fusion feature data corresponding to the first spot image based on the global-neighbor fusion feature data and the global-local fusion feature data.

In an embodiment, the at least one processor may be further configured to execute the one or more instructions to cause the electronic device to segment the neighbor image into a plurality of sub-regions including a first sub-region and a second sub-region. In an embodiment, the at least one processor may be further configured to execute the one or more instructions to cause the electronic device to, by using a trained model, obtain, from the first sub-region, first initial feature data corresponding to the first sub-region, and obtain, from the second sub-region, second initial feature data corresponding to the second sub-region. In an embodiment, the at least one processor may be further configured to execute the one or more instructions to cause the electronic device to obtain the neighbor feature data corresponding to the first spot image based on the first initial feature data and the second initial feature data by using the third artificial intelligence model.

In an embodiment, the at least one processor may be further configured to execute the one or more instructions to cause the electronic device to, by using a trained model, obtain third initial feature data from the first spot image, and obtain fourth initial feature data from the second spot image. In an embodiment, the at least one processor may be further configured to execute the one or more instructions to cause the electronic device to obtain the global feature data corresponding to the first spot image based on the third initial feature data and the fourth initial feature data by using the first artificial intelligence model.

In an embodiment, the first artificial intelligence model may include a positional information encoding model configured to encode positional information in the histology image. In an embodiment, the at least one processor may be further configured to execute the one or more instructions to cause the electronic device to obtain the global feature data corresponding to the first spot image, wherein positional information about the first spot image is encoded in the global feature data, by using the positional information encoding model.

In an embodiment, the first artificial intelligence model, the second artificial intelligence model, the third artificial intelligence model, and the fourth artificial intelligence model may be connected to each other in an end-to-end manner, and trained simultaneously.

In an embodiment, the first artificial intelligence model, the second artificial intelligence model, the third artificial intelligence model, and the fourth artificial intelligence model may be trained by using a loss function based on a difference between a gene expression value predicted for a target spot image included in a training image, and a ground-truth gene expression value for the target spot image.

In an embodiment, the first artificial intelligence model, the second artificial intelligence model, the third artificial intelligence model, and the fourth artificial intelligence model may be trained by using a loss function based on a difference between at least one of a gene expression value predicted from local feature data corresponding to a target spot image included in a training image, a gene expression value predicted from global feature data corresponding to the target spot image, or a gene expression value predicted from neighbor feature data corresponding to the target spot image, and a gene expression value predicted from fusion feature data corresponding to the target spot image.

In an embodiment, the first artificial intelligence model, the second artificial intelligence model, the third artificial intelligence model, and the fourth artificial intelligence model may be trained by using a loss function based on a difference between at least one of a gene expression value predicted from local feature data corresponding to a target spot image included in a training image, a gene expression value predicted from global feature data corresponding to the target spot image, or a gene expression value predicted from neighbor feature data corresponding to the target spot image, and a ground-truth gene expression value for the target spot image.

In an embodiment, a program for performing, on a computer, a method of operating an electronic device may be recorded on a computer-readable recording medium.

A machine-readable storage medium may be provided in the form of a non-transitory storage medium. Here, the term ‘non-transitory storage medium’ refers to a tangible device and does not include a signal (e.g., an electromagnetic wave), and the term ‘non-transitory storage medium’ does not distinguish between a case where data is stored in a storage medium semi-permanently and a case where data is stored temporarily. For example, the ‘non-transitory storage medium’ may include a buffer in which data is temporarily stored.

According to an embodiment, methods according to various embodiments disclosed herein may be included in a computer program product and then provided. The computer program product may be traded as commodities between sellers and buyers. The computer program product may be distributed in the form of a machine-readable storage medium (e.g., a compact disc ROM (CD-ROM)), or may be distributed online (e.g., downloaded or uploaded) through an application store or directly between two user devices (e.g., smart phones). In a case of online distribution, at least a portion of the computer program product (e.g., a downloadable app) may be temporarily stored in a machine-readable storage medium such as a manufacturer's server, an application store's server, or a memory of a relay server.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A method, performed by an electronic device, of predicting gene expression from a histology image by using an artificial intelligence model, the method comprising:
obtaining, based on a first spot image and a second spot image in the histology image, global feature data corresponding to the first spot image by using a first artificial intelligence model;
obtaining, from the first spot image, local feature data corresponding to the first spot image by using a second artificial intelligence model;
obtaining, by using a third artificial intelligence model, neighbor feature data corresponding to the first spot image based on a neighbor image comprising the first spot image and a surrounding region in the histology image; and
predicting gene expression for the first spot image based on the global feature data, the local feature data, and the neighbor feature data by using a fourth artificial intelligence model.

2. The method of claim 1, wherein the predicting of the gene expression for the first spot image based on the global feature data, the local feature data, and the neighbor feature data by using the fourth artificial intelligence model comprises:
obtaining fusion feature data corresponding to the first spot image based on the global feature data, the local feature data, and the neighbor feature data by using a fifth artificial intelligence model; and
predicting the gene expression for the first spot image based on the fusion feature data by using the fourth artificial intelligence model.

3. The method of claim 2, wherein the obtaining of the fusion feature data corresponding to the first spot image comprises:
obtaining, by using the fifth artificial intelligence model, global-neighbor fusion feature data corresponding to the first spot image based on the global feature data and the neighbor feature data, and obtaining, by using the fifth artificial intelligence model, global-local fusion feature data corresponding to the first spot image based on the global feature data and the local feature data; and
obtaining the fusion feature data corresponding to the first spot image based on the global-neighbor fusion feature data and the global-local fusion feature data.

4. The method of claim 1, wherein the obtaining of the neighbor feature data comprises:
segmenting the neighbor image into a plurality of sub-regions comprising a first sub-region and a second sub-region;
obtaining, from the first sub-region by using a trained model, first initial feature data corresponding to the first sub-region, and obtaining, from the second sub-region by using the trained model, second initial feature data corresponding to the second sub-region; and
obtaining the neighbor feature data corresponding to the first spot image based on the first initial feature data and the second initial feature data by using the third artificial intelligence model.

5. The method of claim 1, wherein the obtaining of the global feature data comprises:
obtaining, by using a trained model, third initial feature data from the first spot image, and obtaining, by using the trained model, fourth initial feature data from the second spot image; and
obtaining the global feature data corresponding to the first spot image based on the third initial feature data and the fourth initial feature data by using the first artificial intelligence model.

6. The method of claim 1, wherein the first artificial intelligence model comprises a positional information encoding model configured to encode positional information in the histology image, and
the obtaining of the global feature data comprises obtaining the global feature data corresponding to the first spot image, wherein positional information about the first spot image is encoded in the global feature data, by using the positional information encoding model.

7. The method of claim 1, wherein the first artificial intelligence model, the second artificial intelligence model, the third artificial intelligence model, and the fourth artificial intelligence model are connected to each other in an end-to-end manner and trained simultaneously.

8. The method of claim 1, wherein the first artificial intelligence model, the second artificial intelligence model, the third artificial intelligence model, and the fourth artificial intelligence model are trained by using a loss function based on a difference between a gene expression value predicted for a target spot image included in a training image and a ground-truth gene expression value for the target spot image.

9. The method of claim 1, wherein the first artificial intelligence model, the second artificial intelligence model, the third artificial intelligence model, and the fourth artificial intelligence model are trained by using a loss function based on a difference between at least one of a gene expression value predicted from local feature data corresponding to a target spot image included in a training image, a gene expression value predicted from global feature data corresponding to the target spot image, or a gene expression value predicted from neighbor feature data corresponding to the target spot image and a gene expression value predicted from fusion feature data corresponding to the target spot image.

10. The method of claim 1, wherein the first artificial intelligence model, the second artificial intelligence model, the third artificial intelligence model, and the fourth artificial intelligence model are trained by using a loss function based on a difference between at least one of a gene expression value predicted from local feature data corresponding to a target spot image included in a training image, a gene expression value predicted from global feature data corresponding to the target spot image, or a gene expression value predicted from neighbor feature data corresponding to the target spot image and a ground-truth gene expression value for the target spot image.

11. A computer-readable recording medium having recorded thereon a program for executing, on a computer, the method of claim 1.

12. An electronic device for predicting gene expression from a histology image by using an artificial intelligence model, the electronic device comprising:
a memory storing one or more instructions; and
at least one processor configured to execute the one or more instructions stored in the memory to cause the electronic device to obtain, based on a first spot image and a second spot image in the histology image, global feature data corresponding to the first spot image by using a first artificial intelligence model, obtain, from the first spot image, local feature data corresponding to the first spot image by using a second artificial intelligence model, obtain, by using a third artificial intelligence model, neighbor feature data corresponding to the first spot image based on a neighbor image comprising the first spot image and a surrounding region in the histology image, and predict gene expression for the first spot image based on the global feature data, the local feature data, and the neighbor feature data by using a fourth artificial intelligence model.

13. The electronic device of claim 12, wherein the at least one processor is further configured to execute the one or more instructions to cause the electronic device to obtain fusion feature data corresponding to the first spot image based on the global feature data, the local feature data, and the neighbor feature data by using a fifth artificial intelligence model, and predict the gene expression for the first spot image based on the fusion feature data by using the fourth artificial intelligence model.

14. The electronic device of claim 13, wherein the at least one processor is further configured to execute the one or more instructions to cause the electronic device to obtain, by using the fifth artificial intelligence model, global-neighbor fusion feature data corresponding to the first spot image based on the global feature data and the neighbor feature data, and obtain, by using the fifth artificial intelligence model, global-local fusion feature data corresponding to the first spot image based on the global feature data and the local feature data, and obtain the fusion feature data corresponding to the first spot image based on the global-neighbor fusion feature data and the global-local fusion feature data.

15. The electronic device of claim 12, wherein the at least one processor is further configured to execute the one or more instructions to cause the electronic device to segment the neighbor image into a plurality of sub-regions comprising a first sub-region and a second sub-region, obtain, from the first sub-region by using a trained model, first initial feature data corresponding to the first sub-region, obtain, from the second sub-region by using the trained model, second initial feature data corresponding to the second sub-region, and obtain the neighbor feature data corresponding to the first spot image based on the first initial feature data and the second initial feature data by using the third artificial intelligence model.

16. The electronic device of claim 12, wherein the at least one processor is further configured to execute the one or more instructions to cause the electronic device to, obtain, by using a trained model, third initial feature data from the first spot image, obtain, by using the trained model, fourth initial feature data from the second spot image, and obtain the global feature data corresponding to the first spot image based on the third initial feature data and the fourth initial feature data by using the first artificial intelligence model.

17. The electronic device of claim 12, wherein the first artificial intelligence model comprises a positional information encoding model configured to encode positional information in the histology image, and
the at least one processor is further configured to execute the one or more instructions to cause the electronic device to obtain the global feature data corresponding to the first spot image, wherein positional information about the first spot image is encoded in the global feature data, by using the positional information encoding model.

18. The electronic device of claim 12, wherein the first artificial intelligence model, the second artificial intelligence model, the third artificial intelligence model, and the fourth artificial intelligence model are trained by using a loss function based on a difference between a gene expression value predicted for a target spot image included in a training image and a ground-truth gene expression value for the target spot image.

19. The electronic device of claim 12, wherein the first artificial intelligence model, the second artificial intelligence model, the third artificial intelligence model, and the fourth artificial intelligence model are trained by using a loss function based on a difference between at least one of a gene expression value predicted from local feature data corresponding to a target spot image included in a training image, a gene expression value predicted from global feature data corresponding to the target spot image, or a gene expression value predicted from neighbor feature data corresponding to the target spot image and a gene expression value predicted from fusion feature data corresponding to the target spot image.

20. The electronic device of claim 12, wherein the first artificial intelligence model, the second artificial intelligence model, the third artificial intelligence model, and the fourth artificial intelligence model are trained by using a loss function based on a difference between at least one of a gene expression value predicted from local feature data corresponding to a target spot image included in a training image, a gene expression value predicted from global feature data corresponding to the target spot image, or a gene expression value predicted from neighbor feature data corresponding to the target spot image and a ground-truth gene expression value for the target spot image.
